(19) United States
(12) Patent Application Publication
(10) Pub. No.: US 2024/0157322 A1
(43) Pub. Date: May 16, 2024

(54) CROSSLINKED CORE-SHELL MICROCAPSULES

(71) Applicant: Firmenich SA, Satigny (CH)

(72) Inventor: Yongtao WU, Shanghai (CN)

(21) Appl. No.: 18/281,893

(22) PCT Filed: Mar. 28, 2022
(86) PCT No.: PCT/EP22/58073

§ 371 Date: Sep. 13, 2023

(30) Foreign Application Priority Data
Mar. 30, 2021 (WO) PCT/CN21/84028
May 7, 2021 (EP) 21172651.8

(51) Int. Cl. B01J 13/16; A61K 8/04; A61K 8/11; A61K 8/81; A61K 8/84; A61K 8/895; A61Q 5/02; A61Q 15/00; A61Q 19/10; C11D 3/00; C11D 3/37; C11D 3/50; C11D 17/00

(52) U.S. Cl. CPC B01J 13/16; A61K 8/044; A61K 8/11; A61K 8/8152; A61K 8/84; A61K 8/895; A61Q 5/02; A61Q 15/00; A61Q 19/10; C11D 3/0015; C11D 3/3703; C11D 3/373; C11D 3/3761; C11D 3/505; C11D 17/0013; C11D 17/0039

(57) ABSTRACT

Described herein are a method for preparing a crosslinked core-shell microcapsule slurry, the core-shell microcapsules and their application in perfuming compositions and perfumed consumer products.

### TECHNICAL FIELD OF THE INVENTION

The present invention relates to crosslinked core-shell microcapsule slurry by a new method of preparing it, the core-shell microcapsules per se as well as their application in perfuming compositions and perfumed consumer products.

### BACKGROUND OF THE INVENTION

One of the problems faced by the perfumery industry lies in the relatively rapid loss of olfactive benefit provided by odoriferous compounds due to their volatility, particularly that of “top-notes”. In order to tailor the release rates of volatiles, delivery systems such as microcapsules containing a perfume are needed to protect and later release the core payload when triggered. A key requirement from the industry regarding these systems is to survive suspension in challenging bases without physically dissociating or degrading. This is referred to as stability for the delivery system. For instance, fragranced personal and household cleansers containing high levels of aggressive surfactant detergents are very challenging for the stability of microcapsules.

Polyurea and polyurethane-based microcapsule slurry are widely used for example in perfumery industry for instance as they provide a long lasting pleasant olfactory effect after their applications on different substrates. Those microcapsules have been widely disclosed in the prior art (see for example WO 2007/004166 or EP 2300146 from the Applicant).

Moreover, in addition to the performance in terms of stability and olfactive performance, the consumer demand for eco-friendly delivery systems is more and more important and is driving the development of new delivery systems.

There is therefore still a need to provide new microcapsules using more eco-friendly materials, while not compromising on the performance of the microcapsules, in particular in terms of stability in a challenging medium such as a consumer product base, as well as in delivering a good performance in terms of active ingredient delivery, e.g. olfactive performance in the case of perfuming ingredients.

The present invention is proposing a solution to the above-mentioned problem by providing new microcapsules with dual crosslinked polymeric shell and a process for preparing said microcapsules.

### DETAILED DESCRIPTION OF THE INVENTION

Unless stated otherwise, percentages (%) are meant to designate percent by weight of a composition.

By “hydrophobic material”, it is meant a material which forms a two-phase dispersion when mixed with water. According to the invention, the hydrophobic material can be “inert” material like solvents or active ingredients. According to an embodiment, the hydrophobic material is a hydrophobic active ingredient.

By “active ingredient”, it is meant a single compound or a combination of ingredients.

By “perfume oil”, it is meant a single perfuming or a mixture of several perfuming compounds.

By “consumer product” or “end-product” it is meant a manufactured product ready to be distributed, sold and used by a consumer.

A “microcapsule”, or the similar, in the present invention has a morphology that can vary from a core-shell to a matrix type. According to one embodiment, it is of the core-shell type. In this case, the microcapsules comprise a core based on a hydrophobic material, typically a perfume, and cross-linked polymeric shell surrounding the oil core. Indeed, according to the invention, a dual cross-linked shell is obtained via the polymerization of the multifunctional ethylenically unsaturated monomer, preferably the (meth)acrylate monomer and/or vinyl monomer, and the reaction between the multifunctional ethylenically unsaturated monomer, preferably the (meth)acrylate monomer and/or vinyl monomer, and the multifunctional nucleophile monomer.

Microcapsules have a microcapsule size distribution in the micron range (e.g. a mean diameter) comprised between about 1 and 3000 microns, preferably comprised between 1 and 1000 microns, more preferably between 1 and 500 microns, and even more preferably between 5 and 50 microns.

By “particle size” it is meant an average diameter of particles based on size distribution measured by dynamic light scattering (DLS) using Zetasizer Nano ZS equipment from Malvern Instruments Ltd., UK when particles are dispersed into a water phase.

By “microcapsules size” it is meant the volume mean diameter (D[4,3]) of the relevant capsules, capsules suspension as obtained by laser light scattering of a diluted sample in a Malvern Mastersizer 3000.

By “PEG” it is meant polyethylene glycol. A person skilled in the art is aware that PEG is a polyether compound. A person skilled in the art is aware that PEG also refers to the analogously used terms polyethylene oxide (PEO) or polyoxyethylene (POE).

The present invention relates to a method of preparing a core-shell microcapsule slurry, wherein the process comprises the steps of:
a. Dissolving a multifunctional ethylenically unsaturated monomer, preferably a multifunctional (meth)acrylate monomer and/or a multifunctional vinyl monomer and, optionally, a multifunctional nucleophile monomer in an oil phase comprising a hydrophobic material, preferably a perfume oil, to form an oil phase
b. Preparing an aqueous solution of a stabilizer and, optionally, a multifunctional nucleophile monomer to form a water phase,
c. Adding the oil phase to the water phase to form an oil-in-water emulsion,
d. Optionally, adding a multifunctional nucleophile monomer to the oil-in-water emulsion,
e. Applying conditions to form a crosslinked polymeric shell via the polymerization of the multifunctional ethylenically unsaturated monomer, preferably the (meth)acrylate monomer and/or vinyl monomer, and the reaction between the multifunctional ethylenically unsaturated monomer, preferably the (meth)acrylate monomer and/or vinyl monomer, and the multifunctional nucleophile monomer,
wherein the multifunctional nucleophile monomer is added at least in one of steps (a), (b) or (d).

According to the present invention, a multifunctional ethylenically unsaturated monomer, preferably a multifunctional (meth)acrylate monomer and/or a multifunctional vinyl monomer and, optionally, a multifunctional nucleophile monomer is dissolved in an oil phase comprising a hydrophobic material, preferably a perfume oil, to form an oil phase.

### Multifunctional Ethylenically Unsaturated Monomer

The expression multifunctional ethylenically unsaturated monomer is herein understood as a monomer containing two or more polymerizable ethylenically unsaturated groups.

In a particular embodiment, the multifunctional ethylenically unsaturated monomer is a multifunctional (meth)acrylate monomer.

The expression multifunctional (meth)acrylate monomer is herein understood as a monomer containing two or more polymerizable methacrylate and/or acrylate groups. In a particular embodiment, the multifunctional (meth)acrylate monomer is a monomer containing two or more polymerizable methacrylate groups or two or more acrylate groups. In a particular embodiment, the multifunctional (meth)acrylate monomer is a monomer containing two or more polymerizable acrylate groups.

In a particular embodiment, the (meth)acrylate monomer comprises at least two (meth)acrylate groups, preferably at least three (meth)acrylate groups, preferably four (meth)acrylate groups.

In a particular embodiment, the multifunctional (meth)acrylate monomer is selected from the group consisting of, pentaerythritol tri(meth)acrylate, trimethylolpropane tri(meth)acrylate, pentaerytrithol tetra(meth)acrylate, Tetra(ethylene glycol) di(meth)acrylate, dipentaerytrhitol penta(meth)acryalate, dipentaerytrithol hexa(meth)acrylate, tricyclodecane dimenthanol di(meth)acrylate, ethylene glycol di(meth)acrylate, di(ethylene glycol) di(meth)acrylate, triethylene glycol di(meth)acrylate, propylene glycol di(meth)acrylate, 1,4-butanediol di(meth)acrylate, 1,6-hexanediol di(meth)acrylate, glycerol di(meth)acrylate, 1,3-butanediol di(meth)acrylate, neopentyl glycol di(meth)acrylate, 1,10-decanediol di(meth)acrylate, pentaerythritol tri(meth)acrylate, pentaerythritol tetra(meth)acrylate, dipentaerythritol hexa(meth)acrylate, triallylformal tri(meth)acrylate, allyl methacrylate, trimethylol propane tri(meth)acrylate, tributanediol di(meth)acrylate, PEG 200 di(meth)acrylate, PEG 400 di(meth)acrylate, PEG 600 di(meth)acrylate, pentaerythritol-tetraacrylate, pentaerythritol triacrylate (PETIA), 1,4-butanediol diacrylate (BDA-2), ethylene glycol dimethacrylate, trimethylolpropane triacrylate, hexane diol diacrylate, ((2,4,6-trioxocyclohexane-1,3,5-triyl)tris(oxy))tris(ethane-2,1-diyl) triacrylate, tris(2-acryloyloxyethyl) Isocyanurate, 1,3,5-triacryloylhexahydro-1,3,5-triazine, bis[2-(meth)acryloyloxyethyl]phosphate, bis[glyceryl di(meth)acrylate] phosphate, urethane acrylate oligomers with two to six acrylate groups, polyester/polyether acrylates with more than two acrylate groups, epoxy acrylates with more than two acrylate groups or mixtures thereof.

In a particular embodiment, the multifunctional ethylenically unsaturated monomer is a multifunctional vinyl monomer.

The expression multifunctional vinyl monomer is herein understood as a monomer containing two or more polymerizable vinyl groups.

In a particular embodiment, the multifunctional vinyl monomer comprises at least two vinyl groups, preferably at least three vinyl groups, preferably four vinyl groups.

In a particular embodiment, the multifunctional vinyl monomer is selected from diethylene glycol divinyl ether, 1,5-hexadiene, divinyl adipate, diallyl phthalate, 2,4,6-trimethyl-2,4,6-trivinylcyclotrisiloxane, triallyl phosphate, diallylamine, allyl sulfide, 1,3-divinyltetramethyldisiloxane, divinyl sulfone, tetraallyloxyethane, 1,3,5-trivinyl-1,1,3,5,5-pentamethyltrisiloxane, diallyl isophthalate, allyl ether, triallyl isocyanurate, 1,3-diisopropenylbenzene, 2,2-bis(allyloxymethyl)-1-butanol, diethyl diallylmalonate, 1,2,4-trivinylcyclohexane, triallylamine, diallyl adipate, triallyl cyanurate, diallyl maleate, diallyl terephthalate, 1,3-diisopropenylbenzene, diallyl 1,4-cyclohexanedicarboxylate, bis(vinylsulfonyl)methane, 1,4-cyclohexanedimethanol divinyl ether, di(ethylene glycol) divinyl ether, tri(ethylene glycol) divinyl ether or mixtures thereof.

The multifunctional ethylenically unsaturated monomer, preferably a multifunctional (meth)acrylate monomer and/or a multifunctional vinyl monomer, is preferably comprised in an amount of 0.1 to 20 wt. %, preferably 0.2 to 10 wt. %, more preferably 0.5 to 7 wt. %, based on the total weight of the emulsion obtained after c) or d).

### Multifunctional Nucleophile Monomer

The expression multifunctional nucleophile monomer is herein understood as a monomer containing two or more nucleophilic groups selected from thiols, amines, acetoacetates or mixtures thereof, or is a silane containing at least one nucleophilic group, such as thiol, amine or acetoacetate. The nucleophilic group is able to react with ethylenically unsaturated monomer by forming a new chemical bond.

In an embodiment, the multifunctional nucleophile monomer is a multifunctional thiol, multifunctional amine or multifunctional acetoacetate, comprising two or more groups selected from thiols, amines, acetoacetates or mixtures thereof.

In a particular embodiment, the multifunctional nucleophile monomer is a multifunctional thiol or multifunctional acetoacetate, comprising two or more groups selected from thiols, acetoacetates or mixtures thereof.

In a particular embodiment, the multifunctional nucleophile monomer comprises at least one nucleophilic thiol group and at least one further reactive functional group being selected from silanes, thiols, amines or acetoacetates.

In a particular embodiment, the multifunctional nucleophile monomer is selected from multifunctional thiol monomers comprising two or more thiol groups or one thiol group and a further reactive functional group, such as trimethylolpropane tris(3)-mercaptopropionate), 1,6-hexanedithiol, 2,2′-thiodiethanethiol, 2-amino-1,3,5-triazine-4,6-dithiol, 1,3-propanedithiol, 1,4-butanedithiol, 2,2′-(ethylenedioxy)diethanethiol, benzene-1,4-dithiol, toluene-3,4-dithiol, ethylene glycol bis-mercaptoacetate, ethylene bis(3-mercaptopropionate), 1,4-butanediol bis(thioglycolate), dithiothreitol, pentaerythritol tetra (3-mercaptopropionate), tris[2-(3-mercaptopropionyloxy)ethyl] isocyanurate, trimethylolpropane tris(thioglycolate) or thiosilane monomers, such as 3-mercaptopropyltrimethoxysilane, 3-mercaptopropyltriethoxysilane, (3-mercaptopropyl)methyldimethoxysilane, 11-mercaptoundecyltrimethoxysilane or mixtures thereof.

In a particular embodiment, the multifunctional nucleophile monomer is selected from multifunctional amine monomers consisting of two or more amines, such as ethylenediamine, 1 2-diaminopropane, 1,3-diaminopropane, 1,4-diaminobutane, 1,5-diaminopentane, 1,7-diaminoheptane, 1,8-diaminooctane, 1,9-diaminononane, 1,10-diaminodecane, 1,12-diaminododecane, hexamethylenediamine, phenylenediamine, diaminotoluene, 4-aminobenzylamine, xylylenediamine, triethylenetetramine, diethylenetriamine, spermidine, spermine, agmatine, tris(2-aminoethyl)amine, guanidine carbonate, 3,5-diamino-1,2,4triazole, 2,2,4(2,4,4)-trimethyl-1,6-hexanediamine, 1,3-cyclohexanebis(methylamine), N,N′-bis(3-aminopropyl)ethylenediamine, 1,3-diamino-2-hydroxypropane, 2,2′(ethylenedioxy)diethylamine, aminoguanidine bicarbonate, biguanide, cystamine, 1,1,1-tris(aminomethyl)ethane, polyethylenimine, polyetheramines, polyvinylamine, amino acid (e.g., lysine, cystine, glutamine, arginine), chitosan, protein (e.g., whey protein, caseinate, silk fibroin) or mixtures thereof.

In a particular embodiment, the multifunctional nucleophile monomer is selected from the multifunctional acetoacetate monomers consisting of two or more acetoacetates, such as ethyl diacetoacetate, 1,3-butanediol diacetoacetate, ethylene diacetoacetate, titanium diisopropoxide bis(ethyl acetoacetate), ethyl 2,2′-(4-methoxybenzal)bis-acetoacetate, neopentylglycolycol bis acetoacetate, ethylene glycol diacetoacetate, trimethylolpropane triacetoacetate, pentaerythritol tetraacetoacetate, acetoacetate functionalized cellulose, acetoacetate functionalized starch or mixtures thereof.

The multifunctional nucleophile monomer is preferably comprised in an amount of 0.01 to 20 wt. %, preferably 0.1 to 10 wt. %, more preferably 0.3 to 7 wt. %, based on the total weight of the emulsion obtained after step c) or d).

### Hydrophobic Material

By oil or oil phase it is understood an organic phase that is liquid at about 20° C. which forms the core of the core-shell capsules.

The hydrophobic material according to the invention can be “inert” material like solvents or active ingredients.

When hydrophobic materials are active ingredients, they are preferably chosen from the group consisting of flavors, flavor ingredients, perfumes, perfume ingredients, nutraceuticals, cosmetics, pest control agents, biocide actives and mixtures thereof.

According to a particular embodiment, the hydrophobic material comprises a mixture of a perfume with another ingredient selected from the group consisting of nutraceuticals, cosmetics, pest control agents and biocide actives.

According to a particular embodiment, the hydrophobic material comprises a mixture of biocide actives with another ingredient selected from the group consisting of perfumes, nutraceuticals, cosmetics, pest control agents.

According to a particular embodiment, the hydrophobic material comprises a mixture of pest control agents with another ingredient selected from the group consisting of perfumes, nutraceuticals, cosmetics, biocide actives.

According to a particular embodiment, the hydrophobic material comprises a perfume.

According to a particular embodiment, the hydrophobic material consists of a perfume.

According to a particular embodiment, the hydrophobic material consists of biocide actives.

According to a particular embodiment, the hydrophobic material consists of pest control agents.

By “perfume” (or also “perfume oil”) what is meant here is an ingredient or a composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odor. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes a combination of perfuming ingredients with substances which together improve, enhance or modify the delivery of the perfuming ingredients, such as perfume precursors, emulsions or dispersions, as well as combinations which impart an additional benefit beyond that of modifying or imparting an odor, such as long-lastingness, blooming, malodor counteraction, antimicrobial effect, microbial stability, pest control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulfurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

In particular one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0-2,7-]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl)propanal, diethyl 1,4-cyclohexanedicarboxylate, 3-methyl-2-hexen-1-yl acetate, 1-[3,3-dimethylcyclohexyl]ethyl[3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.0^{2,7}]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl)butanal.

According to a particular embodiment, the perfume or perfume formulation comprises a fragrance modulator (that can be used in addition to the hydrophobic solvent when present or as substitution of the hydrophobic solvent when there is no hydrophobic solvent).

Preferably, the fragrance modulator is defined as a fragrance material with
i. a vapor pressure of less than 0.0008 Torr at 22° C.;
ii. a clog P of 3.5 and higher, preferably 4.0 and higher and more preferably 4.5
iii. at least two Hansen solubility parameters selected from a first group consisting of: an atomic dispersion force from 12 to 20, a dipole moment from 1 to 7, and a hydrogen bonding from 2.5 to 11,
iv. at least two Hansen solubility parameters selected from a second group consisting of: an atomic dispersion force from 14 to 20, a dipole moment from 1 to 8, and a hydrogen bonding from 4 to 11, when in solution with a compound having a vapor pressure range of 0.0008 to 0.08 Torr at 22° C.

Preferably as examples the following ingredients can be listed as fragrance modulators but the list in not limited to the following materials: alcohol C12, oxacyclohexadec-12/13-en-2-one, 3-[(2′,2′,3′-trimethyl-3′-cyclopenten-1′-yl)methoxy]-2-butanol, cyclohexadecanone, (Z)-4-cyclopentadecen-1-one, cyclopentadecanone, (8Z)-oxacycloheptadec-8-en-2-one, 2-[5-(tetrahydro-5-methyl-5-vinyl-2-furyl)-tetrahydro-5-methyl-2-furyl]-2-propanol, muguet aldehyde, 1,5,8-trimethyl-13-oxabicyclo[10.1.0]trideca-4,8-diene, (+−)-4,6,6,7,8,8-hexamethyl-1,3,4,6,7,8-hexahydrocyclopenta[g]isochromene, (+)-(1S,2S,3S,5R)-2,6,6-trimethylspiro[bicyclo[3.1.1]heptane-3,1′-cyclohexane]-2′-en-4′-one, oxacyclohexadecan-2-one, 2-{(1S)-1-[(1R)-3,3-dimethylcyclohexyl]ethoxy}-2-oxoethyl propionate, (+)-(4R,4aS,6R)-4,4a-dimethyl-6-(1-propen-2-yl)-4,4a,5,6,7,8-hexahydro-2(3H)-naphthalenone, amylcinnamic aldehyde, hexylcinnamic aldehyde, hexyl salicylate, (1E)-1-(2,6,6-trimethyl-1-cyclohexen-1-yl)-1,6-heptadien-3-one, (9Z)-9-cycloheptadecen-1-one.

It is also understood that said ingredients may also be compounds known to release in a controlled manner various types of perfuming compounds also known as properfume or profragrance. Non-limiting examples of suitable proper fumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

According to a particular embodiment, the perfume comprises at least 35% of perfuming ingredients having a log P above 3.

Log P is the common logarithm of estimated octanol-water partition coefficient, which is known as a measure of lipophilicity.

The Log P values of many perfuming compound have been reported, for example, in the Pomona92 database, available from Daylight Chemical Information Systems, Inc. (Daylight CIS), Irvine, Calif., which also contains citations to the original literature. Log P values are most conveniently calculated by the “CLOGP” program, also available from Daylight CIS. This program also lists experimental log P values when they are available in the Pomona92 database. The “calculated log P” (cLog P) is determined by the fragment approach of Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990). The fragment approach is based on the chemical structure of each perfume oil ingredient, and takes into account the numbers and types of atoms, the atom connectivity, and chemical bonding. The cLog P values, which are the most reliable and widely used estimates for this physicochemical property, are preferably used instead of the experimental Log P values in the selection of perfuming compounds which are useful in the present invention.

In a particular embodiment, the perfume oil comprises at least 40 wt. %, preferably at least 50 wt. %, more preferably at least 60 wt. % of ingredients having a log P above 3, preferably above 3.5 and even more preferably above 3.75.

Preferably, the perfume oil contains less than 10 wt. % of its own weight of primary alcohols, less than 15 wt. % of its own weight of secondary alcohols and less than 20% of its own weight of tertiary alcohols. Advantageously, the perfume used in the invention does not contain any primary alcohols and contains less than 15 wt. % of secondary and tertiary alcohols.

According to a particular embodiment, the perfume comprises at least 20 wt. %, preferably at least 25 wt. %, more preferably at least 40 wt. % of Bulky materials of groups 1 to 6, preferably 3 to 6.

The term Bulky materials is herein understood as perfuming ingredients having a high steric hindrance, i.e. having a substitution pattern which provides high steric hindrance and thus the Bulky materials are in particular those from one of the following groups:
Group 1: perfuming ingredients comprising a cyclohexane, cyclohexene, cyclohexanone or cyclohexenone ring substituted with at least one 1 to 4 nodes comprising substituent, preferably at least one linear or branched C₁ to C₄ alkyl or alkenyl substituent;
Group 2: perfuming ingredients comprising a cyclopentane, cyclopentene, cyclopentanone or cyclopentenone ring substituted with at least one 4 or more nodes comprising substituent, preferably at least one linear or branched C₄ or longer, preferably C₄ to C₈ alkyl or alkenyl substituent;
Group 3: perfuming ingredients comprising a phenyl ring or perfuming ingredients comprising a cyclohexane, cyclohexene, cyclohexanone or cyclohexenone ring substituted with at least one 5 or more nodes comprising substituent, preferably at least one linear or branched C₅ or longer, preferably C₅ to C₈, alkyl or alkenyl substituent, or with at least one phenyl substituent and optionally one or more 1 to 3 nodes comprising substituents, preferably one or more linear or branched C₁ to C₃ alkyl or alkenyl substituents;
Group 4: perfuming ingredients comprising at least two fused or linked 5 membered or 6 membered rings, preferably at least two fused or linked C₅ and/or C₆ rings;
Group 5: perfuming ingredients comprising a camphor-like ring structure, i.e. two 5 or 6 membered rings that are fused in a bridge-type fashion;
Group 6: perfuming ingredients comprising at least one 7 to 20 membered ring, preferably at least one C7 or C20 ring structure.

The term nodes as understood in this context means any atom which is able to provide at least two, preferably at least 3, more preferably 4, bonds to further atoms. Particular examples of nodes as herein understood are carbon atoms (up to 4 bonds to further atoms), nitrogen atoms (up to 3 bonds to further atoms), oxygen atoms (up to 2 bonds to further atoms) and sulfur (up to 2 bonds to further atoms). Particular examples of further atoms as understood in this context could be carbon atoms, nitrogen atoms, sulfur atoms, oxygen atoms and hydrogen atoms.

Examples of ingredients from each of these groups are:
Group 1: 2,4-dimethyl-3-cyclohexene-1-carbaldehyde (origin: Firmenich S A, Geneva, Switzerland), isocyclocitral, menthone, isomenthone, methyl 2,2-dimethyl-6-methylene-1-cyclohexanecarboxylate (origin: Firmenich S A, Geneva, Switzerland), nerone, terpineol, dihydroterpineol, terpenyl acetate, dihydroterpenyl acetate, dipentene, eucalyptol, hexylate, rose oxide, (S)-1,8-p-menthadiene-7-ol (origin: Firmenich S A, Geneva, Switzerland), 1-p-menthene-4-ol, (1RS,3RS, 4SR)-3-p-mentanyl acetate, (1R,2S,4R)-4,6,6-trimethyl-bicyclo[3,1,1]heptan-2-ol, tetrahydro-4-methyl-2-phenyl-2H-pyran (origin: Firmenich S A, Geneva, Switzerland), cyclohexyl acetate, cyclanol acetate, 1,4-cyclohexane diethyldicarboxylate (origin: Firmenich S A, Geneva, Switzerland), (3ARS, 6SR, 7ASR)-perhydro-3,6-dimethyl-benzo[B]furan-2-one (origin: Firmenich S A, Geneva, Switzerland), ((6R)-perhydro-3,6-dimethyl-benzo[B]furan-2-one (origin: Firmenich S A, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan S A, Vernier, Switzerland), (1′R,E)-2-ethyl-4-(2′,2′,3′-trimethyl-3′-cyclopenten-1′-yl)-2-buten-1-ol (origin: Firmenich S A, Geneva, Switzerland), (1′R,E)-3,3-dimethyl-5-(2′,2′,3′-trimethyl-3′-cyclopenten-1′-yl)-4-penten-2-ol (origin: Firmenich S A, Geneva, Switzerland), 2-heptylcyclopentanone, methyl-cis-3-oxo-2-pentyl-1-cyclopentane acetate (origin: Firmenich S A, Geneva, Switzerland), 2,2,5-Trimethyl-5-pentyl-1-cyclopentanone (origin: Firmenich S A, Geneva, Switzerland), 3,3-dimethyl-5-(2,2,3-trimethyl-3-cyclopenten-1-yl)-4-penten-2-ol (origin: Firmenich S A, Geneva, Switzerland), 3-methyl-5-(2,2,3-trimethyl-3-cyclopenten-1-yl)-2-pentanol (origin, Givaudan S A, Vernier, Switzerland);
Group 3: damascones, 1-(5,5-dimethyl-1-cyclohexen-1-yl)-4-penten-1-one (origin: Firmenich S A, Geneva, Switzerland), nectalactone ((1′R)-2-[2-(4′-methyl-3′-cyclohexen-1′-yl)propyl]cyclopentanone), alpha-ionone, beta-ionone, damascenone, mixture of 1-(5,5-dimethyl-1-cyclohexen-1-yl)-4-penten-1-one and 1-(3,3-dimethyl-1-cyclohexen-1-yl)-4-penten-1-one (origin: Firmenich S A, Geneva, Switzerland), 1-(2,6,6-trimethyl-1-cyclohexen-1-yl)-2-buten-1-one (origin: Firmenich S A, Geneva, Switzerland), (1S,1′R)-[1-(3′,3′-Dimethyl-1′-cyclohexyl)ethoxycarbonyl]methyl propanoate (origin: Firmenich S A, Geneva, Switzerland), 2-tert-butyl-1-cyclohexyl acetate (origin: International Flavors and Fragrances, USA), 1-(2,2,3,6-tetramethyl-cyclohexyl)-3-hexanol (origin: Firmenich S A, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, 4-(1,1-dimethylethyl)-1-cyclohexyl acetate (origin: Firmenich SA, Geneva, Switzerland), 8-methoxy-1-p-menthene, (1S,1′R)-2-[1-(3′,3′-dimethyl-1′-cyclohexyl) ethoxy]-2-methylpropyl propanoate (origin: Firmenich S A, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate, 2-methoxy-4-methylphenyl methyl carbonate, ethyl 2-methoxy-4-methylphenyl carbonate, 4-ethyl-2-methoxyphenyl methyl carbonate;
Group 4: Methyl cedryl ketone (origin: International Flavors and Fragrances, USA), a mixture of (1RS,2SR, 6RS,7RS, 8SR)-tricyclo[5.2.1.0-2,6-]dec-3-en-8-yl 2-methylpropanoate and (1 RS,2SR, 6RS,7RS, 8SR)-tricyclo[5.2.1.0-2,6-]dec-4-en-8-yl 2-methylpropanoate, vetyverol, vetyverone, 1-(octahydro-2,3,8,8-tetramethyl-2-naphtalenyl)-1-ethanone (origin: International Flavors and Fragrances, USA), (5RS,9RS, 10SR)-2,6,9,10-tetramethyl-1-oxaspiro[4.5]deca-3,6-diene and the (5RS,9SR, 10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), a mixture of 3-(3,3-dimethyl-5-indanyl)propanal and 3-(1,1-dimethyl-5-indanyl)propanal (origin: Firmenich S A, Geneva, Switzerland), 3′,4-dimethyl-tricyclo[6.2.1.0(2,7)]undec-4-ene-9-spiro-2′-oxirane (origin: Firmenich S A, Geneva, Switzerland), 9/10-ethyldiene-3-oxatricyclo[6.2.1.0(2,7)]undecane, (perhydro-5,5,8A-trimethyl-2-naphthalenyl acetate (origin: Firmenich S A, Geneva, Switzerland), octalynol, (dodecahydro-3a,6,6,9a-tetramethyl-naphtho[2,1-b]furan, origin: Firmenich S A, Geneva, Switzerland), tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate, (+)-(1S,2S,3S)-2,6,6-trimethyl-bicyclo[3.1.1]heptane-3-spiro-2′-cyclohexen-4′-one;
Group 5: camphor, borneol, isobornyl acetate, 8-isopropyl-6-methyl-bicyclo[2.2.2]oct-5-ene-2-carbaldehyde, pinene, camphene, 8-methoxycedrane, (8-methoxy-2,6,6,8-tetramethyl-tricyclo[5.3.1.0(1,5)]undecane (origin: Firmenich S A, Geneva, Switzerland), cedrene, cedrenol, cedrol, mixture of 9-ethylidene-3-oxatricyclo[6.2.1.0(2,7)]undecan-4-one and 10-ethylidene-3-oxatricyclo[6.2.1.0(2,7)]undecan-4-one (origin: Firmenich S A, Geneva, Switzerland), 3-methoxy-7,7-dimethyl-10-methylene-bicyclo[4.3.1]decane (origin: Firmenich S A, Geneva, Switzerland);
Group 6: (trimethyl-13-oxabicyclo-[10.1.0]-trideca-4,8-diene (origin: Firmenich S A, Geneva, Switzerland), Ambrettolide LG ((E)-9-hexadecen-16-olide, origin: Firmenich S A, Geneva, Switzerland), pentadecenolide (origin: Firmenich S A, Geneva, Switzerland), muscenone (3-methyl-(4/5)-cyclopentadecenone, origin: Firmenich S A, Geneva, Switzerland), 3-methylcyclopentadecanone (origin: Firmenich S A, Geneva, Switzerland), pentadecanolide (origin: Firmenich S A, Geneva, Switzerland), cyclopentadecanone (origin: Firmenich S A, Geneva, Switzerland), 1-ethoxyethoxy)cyclododecane (origin: Firmenich S A, Geneva, Switzerland), 1,4-dioxacycloheptadecane-5,17-dione, 4,8-cyclododecadien-1-one;
Group 7: (+−)-2-methyl-3-[4-(2-methyl-2-propanyl)phenyl]propanal (origin: Givaudan S A, Vernier, Switzerland), 2,2,2-trichloro-1-phenylethyl acetate.

Preferably, the perfume comprises at least 30%, preferably at least 50%, more preferably at least 60% of ingredients selected from Groups 1 to 7, as defined above. More preferably said perfume comprises at least 30%, preferably at least 50% of ingredients from Groups 3 to 7, as defined above. Most preferably said perfume comprises at least 30%, preferably at least 50% of ingredients from Groups 3, 4, 6 or 7, as defined above.

According to another preferred embodiment, the perfume comprises at least 30%, preferably at least 50%, more preferably at least 60% of ingredients having a log P above 3, preferably above 3.5 and even more preferably above 3.75.

Preferably, the perfume used in the invention contains less than 10% of its own weight of primary alcohols, less than 15% of its own weight of secondary alcohols and less than 20% of its own weight of tertiary alcohols. Advantageously, the perfume used in the invention does not contain any primary alcohols and contains less than 15% of secondary and tertiary alcohols.

According to an embodiment, the oil phase (or the oil core) comprises:
25-100 wt % of a perfume oil comprising at least 15 wt % of high impact perfume raw materials having a Log T<-4, and
0-75 wt % of a density balancing material having a density greater than 1.07 g/cm³.
   “High impact perfume raw materials” should be understood as perfume raw materials having a Log T<-4. The odor threshold concentration of a chemical compound is determined in part by its shape, polarity, partial charges and molecular mass. For convenience, the threshold concentration is presented as the common logarithm of the threshold concentration, i.e., Log [Threshold] (“LogT”).

A “density balancing material” should be understood as a material having a density preferably greater than 1.07 g/cm³ and having preferably low or no odor.

The density of a component is defined as the ratio between its mass and its volume (g/cm³).
Several methods are available to determine the density of a component.
One may refer for example to the ISO 298:1998 method to measure d20 densities of essential oils.

The odor threshold concentration of a perfuming compound is determined by using a gas chromatograph (“GC”). Specifically, the gas chromatograph is calibrated to determine the exact volume of the perfume oil ingredient injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of the perfuming compound. To determine the threshold concentration, solutions are delivered to the sniff port at the back-calculated concentration. A panelist sniffs the GC effluent and identifies the retention time when odor is noticed. The average across all panelists determines the odor threshold concentration of the perfuming compound. The determination of odor threshold is described in more detail in C. Vuilleumier et al., Multidimensional Visualization of Physical and Perceptual Data Leading to a Creative Approach in Fragrance Development, Perfume & Flavorist, Vol. 33, September, 2008, pages 54-61.

According to an embodiment, the high impact perfume raw materials having a Log T<-4 are selected from the group consisting of (+−)-1-methoxy-3-hexanethiol, 4-(4-hydroxy-1-phenyl)-2-butanone, 2-methoxy-4-(1-propenyl)-1-phenyl acetate, pyrazobutyle, 3-propylphenol, 1-(3-methyl-1-benzofuran-2-yl)ethanone, 2-(3-phenylpropyl)pyridine, 1-(3,3/5,5-dimethyl-1-cyclohexen-1-yl)-4-penten-1-one, 1-(5,5-dimethyl-1-cyclohexen-1-yl)-4-penten-1-one, a mixture comprising (3RS,3aRS, 6SR, 7ASR)-perhydro-3,6-dimethyl-benzo[b]furan-2-one and (3SR, 3aRS, 6SR, 7ASR)-perhydro-3,6-dimethyl-benzo[b]furan-2-one, (+−)-1-(5-ethyl-5-methyl-1-cyclohexen-1-yl)-4-penten-1-one, (1'S, 3′R)-1-methyl-2-[(1′,2′,2′-trimethylbicyclo[3.1.0]hex-3′-yl)methyl]cyclopropyl}methanol, (+−)-3-mercaptohexyl acetate, (2E)-1-(2,6,6-trimethyl-1,3-cyclohexadien-1-yl)-2-buten-1-one, H-methyl-2h-1,5-benzodioxepin-3(4H)-one, (2E,6Z)-2,6-nonadien-1-ol, (4Z)-4-dodecenal, (+−)-4-hydroxy-2,5-dimethyl-3(2H)-furanone, methyl 2,4-dihydroxy-3,6-dimethylbenzoate, 3-methylindole, (+−)-perhydro-4alpha, 8abeta-dimethyl-4a-naphthalenol, patchoulol, 2-methoxy-4-(1-propenyl)phenol, mixture comprising (+−)-5,6-dihydro-4-methyl-2-phenyl-2H-pyran and tetrahydro-4-methylene-2-phenyl-2H-pyran, mixture comprising 4-methylene-2-phenyltetrahydro-2H-pyran and (+−)-4-methyl-2-phenyl-3,6-dihydro-2H-pyran, 4-hydroxy-3-methoxybenzaldehyde, nonylenic aldehyde, 2-methoxy-4-propylphenol, 3-methyl-5-phenyl-2-pentenenitrile, 1-(spiro[4.5]dec-6/7-en-7-yl)-4-penten-1-one(, 2-methoxynaphthalene, (−)-(3aR,5AS, 9AS, 9BR)-3a,6,6,9a-tetramethyldodecahydronaphtho[2,1-b]furan, 5-nonanolide, (3aR,5AS, 9AS, 9BR)-3a,6,6,9a-tetramethyldodecahydronaphtho[2,1-b]furan, 7-isopropyl-2H,4H-1,5-benzodioxepin-3-one, coumarin, 4-methylphenyl isobutyrate, (2E)-1-(2,6,6-trimethyl-1,3-cyclohexadien-1-yl)-2-buten-1-one, beta,2,2,3-tetramethyl-delta-methylene-3-cyclopentene-1-butanol, delta damascone ((2E)-1-[(1RS,2SR)-2,6,6-trimethyl-3-cyclohexen-1-yl]-2-buten-1-one), (+−)-3,6-dihydro-4,6-dimethyl-2-phenyl-2h-pyran, anisaldehyde, paracresol, 3-ethoxy-4-hydroxybenzaldehyde, methyl 2-aminobenzoate, ethyl methylphenylglycidate, octalactone gamma, ethyl 3-phenyl-2-propenoate, (−)-(2E)-2-ethyl-4-[(1R)-2,2,3-trimethyl-3-cyclopenten-1-yl]-2-buten-1-ol, paracresyl acetate, dodecalactone, tricyclone, (+)-(3R,5Z)-3-methyl-5-cyclopentadecen-1-one, undecalactone, (1R,4R)-8-mercapto-3-p-menthanone, (3S,3AS,6R,7AR)-3,6-dimethylhexahydro-1-benzofuran-2(3H)-one, beta ionone, (+−)-6-pentyltetrahydro-2H-pyran-2-one, (3E,5Z)-1,3,5-undecatriene, 10-undecenal, (9E)-9-undecenal (9Z)-9-undecenal, (Z)-4-decenal, (+−)-ethyl 2-methylpentanoate, 1,2-diallyldisulfane, 2-tridecenenitrile, 3-tridecenenitrile, (+−)-2-ethyl-4,4-dimethyl-1,3-oxathiane, (+)-(3R,5Z)-3-methyl-5-cyclopentadecen-1-one, 3-(4-tert-butylphenyl)propanal, allyl (cyclohexyloxy)acetate, methylnaphthylketone, (+−)-(4E)-3-methyl-4-cyclopentadecen-1-one, (+−)-5E3-methyl-5-cyclopentadecen-1-one, cyclopropylmethyl 3-hexenoate, (4E)-4-methyl-5-(4-methylphenyl)-4-pentenal, (+−)-1-(5-propyl-1,3-benzodioxol-2-yl)ethanone, 4-methyl-2-pentylpyridine, (+−)-(E)-3-methyl-4-(2,6,6-trimethyl-2-cyclohexen-1-yl)-3-buten-2-one, (3aRS, 5aSR, 9aSR, 9bRS)-3a,6,6,9a-tetramethyldodecahydronaphtho[2,1-b]furan, (2S,5R)-5-methyl-2-(2-propanyl)cyclohexanone oxime, 6-hexyltetrahydro-2H-pyran-2-one, (+−)-3-(3-isopropyl-1-phenyl)butanal, methyl 2-(3-oxo-2-pentylcyclopentyl)acetate, 1-(2,6,6-trimethyl-1-cyclohex-2-enyl)pent-1-en-3-one, indol, 7-propyl-2H,4H-1,5-benzodioxepin-3-one, ethyl praline, (4-methylphenoxy)acetaldehyde, ethyl tricyclo[5.2.1.0.2,6]decane-2-carboxylate, (+)-(1'S,2S,E)-3,3-dimethyl-5-(2′,2′,3′-trimethyl-3′-cyclopenten-1′-yl)-4-penten-2-ol, (4E)-3,3-dimethyl-5-[(1R)-2,2,3-trimethyl-3-cyclopenten-1-yl]-4-penten-2-ol, 8-isopropyl-6-methyl-bicyclo[2.2.2]oct-5-ene-2-carbaldehyde, methylnonylacetaldehyde, 4-formyl-2-methoxyphenyl 2-methylpropanoate, (E)-4-decenal, (+−)-2-ethyl-4-(2,2,3-trimethyl-3-cyclopenten-1-yl)-2-buten-1-ol, (1R,5R)-4,7,7-trimethyl-6-thiabicyclo[3.2.1]oct-3-ene, (1R,4R,5R)-4,7,7-trimethyl-6-thiabicyclo[3.2.1]octane, (−)-(3R)-3,7-dimethyl-1,6-octadien-3-ol, (E)-3-phenyl-2-propenenitrile, 4-methoxybenzyl acetate, (E)-3-methyl-5-(2,2,3-trimethyl-3-cyclopenten-1-yl)-4-penten-2-ol, allyl (2/3-methylbutoxy)acetate, (+−)-(2E)-1-(2,6,6-trimethyl-2-cyclohexen-1-yl)-2-buten-1-one, (1E)-1-(2,6,6-trimethyl-1-cyclohexen-1-yl)-1-penten-3-one, and mixtures thereof.

According to an embodiment, perfume raw materials having a Log T<-4 are chosen in the group consisting of aldehydes, ketones, alcohols, phenols, esters lactones, ethers, epoxydes, nitriles and mixtures thereof.

According to an embodiment, perfume raw materials having a Log T<-4 comprise at least one compound chosen in the group consisting of alcohols, phenols, esters lactones, ethers, epoxydes, nitriles and mixtures thereof, preferably in amount comprised between 20 and 70 wt. % based on the total weight of the perfume raw materials having a Log T<-4.

According to an embodiment, perfume raw materials having a Log T<-4 comprise between 20 and 70 wt. % by weight of aldehydes, ketones, and mixtures thereof based on the total weight of the perfume raw materials having a Log T<-4.

The remaining perfume raw materials contained in the oil core may have therefore a Log T>-4.

According to an embodiment, the perfume raw materials having a Log T>-4 are chosen in the group consisting of ethyl 2-methylbutyrate, (E)-3-phenyl-2-propenyl acetate, (+-)-6/8-sec-butylquinoline, (+−)-3-(1,3-benzodioxol-5-yl)-2-methylpropanal, verdyl propionate, 1-(octahydro-2,3,8,8-tetramethyl-2-naphtalenyl)-1-ethanone, methyl 2-((1 RS,2RS)-3-oxo-2-pentylcyclopentyl)acetate, (+−)-(E)-4-methyl-3-decen-5-ol, 2,4-dimethyl-3-cyclohexene-1-carbaldehyde, 1,3,3-trimethyl-2-oxabicyclo[2.2.2]octane, tetrahydro-4-methyl-2-(2-methyl-1-propenyl)-2H-pyran, dodecanal, 1-oxa-12/13-cyclohexadecen-2-one, (+−)-3-(4-isopropylphenyl)-2-methylpropanal, aldehyde C11, (+−)-2,6-dimethyl-7-octen-2-ol, allyl 3-cyclohexylpropanoate, (Z)-3-hexenyl acetate, 5-methyl-2-(2-propanyl)cyclohexanone, allyl heptanoate, 2-(2-methyl-2-propanyl)cyclohexyl acetate, 1,1-dimethyl-2-phenylethyl butyrate, geranyl acetate, neryl acetate, (+−)-1-phenylethyl acetate, 1,1-dimethyl-2-phenylethyl acetate, 3-methyl-2-butenyl acetate, ethyl 3-oxobutanoate, (2Z)-ethyl 3-hydroxy-2-butenoate, 8-p-menthanol, 8-p-menthanyl acetate, 1-p-menthanyl acetate, (+−)-2-(4-methyl-3-cyclohexen-1-yl)-2-propanyl acetate, (+−)-2-methylbutyl butanoate, 2-{(1S)-1-[(1R)-3,3-dimethylcyclohexyl]ethoxy}-2-oxoethyl propionate, 3,5,6-trimethyl-3-cyclohexene-1-carbaldehyde, 2,4,6-trimethyl-3-cyclohexene-1-carbaldehyde, 2-cyclohexylethyl acetate, octanal, ethyl butanoate, (+−)-(3E)-4-(2,6,6-trimethyl-1/2-cyclohexen-1-yl)-3-buten-2-one, 1-[(1RS,6SR)-2,2,6-trimethylcyclohexyl]-3-hexanol, 1,3,3-trimethyl-2-oxabicyclo[2.2.2]octane, 1,3,3-trimethyl-2-oxabicyclo[2.2.2]octane, ethyl hexanoate, undecanal, decanal, 2-phenylethyl acetate, (1S,2S,4S)-1,7,7-trimethylbicyclo[2.2.1]heptan-2-ol, (1S,2R,4S)-1,7,7-trimethylbicyclo[2.2.1]heptan-2-ol), (+−)-3,7-dimethyl-3-octanol, 1-methyl-4-(2-propanylidene)cyclohexene, (+)-(R)-4-(2-methoxypropan-2-yl)-1-methylcyclohex-1-ene, verdyl acetate, (3R)-1-[(1R,6S)-2,2,6-trimethylcyclohexyl]-3-hexanol, (3S)-1-[(1R,6S)-2,2,6-trimethylcyclohexyl]-3-hexanol, (3R)-1-[(1S,6S)-2,2,6-trimethylcyclohexyl]-3-hexanol, (+)-(1S,1′R)-2-[1-(3′,3′-dimethyl-1′-cyclohexyl)ethoxy]-2-methylpropyl propanoate, and mixtures thereof.

The nature of high impact perfume raw materials having a Log T<-4 and density balancing material having a density greater than 1.07 g/cm³ are described in WO2018115250, the content of which are included by reference.

The term “biocide” refers to a chemical substance capable of killing living organisms (e.g. microorganisms) or reducing or preventing their growth and/or accumulation. Biocides are commonly used in medicine, agriculture, forestry, and in industry where they prevent the fouling of, for example, water, agricultural products including seed, and oil pipelines. A biocide can be a pesticide, including a fungicide, herbicide, insecticide, algicide, molluscicide, miticide and rodenticide; and/or an antimicrobial such as a germicide, antibiotic, antibacterial, antiviral, antifungal, antiprotozoal and/or antiparasite.

As used herein, a “pest control agent” indicates a substance that serves to repel or attract pests, to decrease, inhibit or promote their growth, development or their activity. Pests refer to any living organism, whether animal, plant or fungus, which is invasive or troublesome to plants or animals, pests include insects notably arthropods, mites, spiders, fungi, weeds, bacteria and other microorganisms.

According to an embodiment, the perfume formulation comprises
0 to 60 wt. % of a hydrophobic solvent (based on the total weight of the perfume formulation),
40 to 100 wt. % of a perfume oil (based on the total weight of the perfume formulation), wherein the perfume oil has at least two, preferably all of the following characteristics:
   at least 35%, preferably at least 40%, preferably at least 50%, more preferably at least 60% of perfuming ingredients having a log P above 3, preferably above 3.5,
   at least 20%, preferably at least 25%, preferably at least 30%, more preferably at least 40% of Bulky materials of groups 1 to 6, preferably 3 to 6 as previously defined and
   at least 15%, preferably at least 20%, more preferably at least 25%, even more preferably at least 30% of high impact perfume materials having a Log T<-4 as previously defined,
      optionally, further hydrophobic active ingredients.

According to a particular embodiment, the perfume comprises 0 to 60 wt. % of a hydrophobic solvent.

According to a particular embodiment, the hydrophobic solvent is a density balancing material preferably chosen in the group consisting of benzyl salicylate, benzyl benzoate, cyclohexyl salicylate, benzyl phenylacetate, phenylethyl phenylacetate, triacetin, ethyl citrate, methyl and ethyl salicylate, benzyl cinnamate, and mixtures thereof.

In a particular embodiment, the hydrophobic solvent has Hansen Solubility Parameters compatible with entrapped perfume oil.

The term “Hansen solubility parameter” is understood refers to a solubility parameter approach proposed by Charles Hansen used to predict polymer solubility and was developed around the basis that the total energy of vaporization of a liquid consists of several individual parts. To calculate the “weighted Hansen solubility parameter” one must combine the effects of (atomic) dispersion forces, (molecular) permanent dipole-permanent dipole forces, and (molecular) hydrogen bonding (electron exchange). The weighted Hansen solubility parameter” is calculated as (δD²+δP²+δH²)^{0.5}, wherein δD is the Hansen dispersion value (also referred to in the following as the atomic dispersion fore), δP is the Hansen polarizability value (also referred to in the following as the dipole moment), and δH is the Hansen Hydrogen-bonding (“h-bonding”) value (also referred to in the following as hydrogen bonding). For a more detailed description of the parameters and values, see Charles Hansen, The Three Dimensional Solubility Parameter and Solvent Diffusion Coefficient, Danish Technical Press (Copenhagen, 1967).

Euclidean difference in solubility parameter between a fragrance and a solvent is calculated as (4*(δDₛₒₗᵥₑₙₜδD_{fragrance})²+(δPₛₒₗᵥₑₙₜ−δP_{fragrance})²+(δH_{solvent-δH}_{_{fragrance}}₎²)^{0.5}, in which δDₛₒₗᵥₑₙₜ, δPₛₒₗᵥₑₙₜ, and δHₛₒₗᵥₑₙₜ, are the Hansen dispersion value, Hansen polarizability value, and Hansen h-bonding values of the solvent, respectively; and δD_{fragrance}, δP_{fragrance}, and δH_{fragrance} are the Hansen dispersion value, Hansen polarizability value, and Hansen h-bonding values of the fragrance, respectively.

In a particular embodiment, the perfume oil and the hydrophobic solvent have at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11

According to a particular embodiment, the hydrophobic material is free of any active ingredient (such as perfume). According to this particular embodiment, it comprises, preferably consists of hydrophobic solvents, preferably chosen in the group consisting of isopropyl myristate, triglycerides (e.g. Neobee® MCT oil, vegetable oils), D-limonene, silicone oil, mineral oil, and mixtures thereof with optionally hydrophilic solvents preferably chosen in the group consisting of 1,4-butanediol, benzyl alcohol, triethyl citrate, triacetin, benzyl acetate, ethyl acetate, propylene glycol (1,2-propanediol), 1,3-propanediol, dipropylene glycol, glycerol, glycol ethers and mixtures thereof.

The oil phase comprising a hydrophobic active material, preferably a perfume oil, is preferably comprised in an amount of 1 to 60 wt. %, preferably 10 to 50 wt. %, more preferably 15 to 40 wt. %, based on the total weight of the emulsion obtained after c) or d).

According to the present invention, an aqueous solution of a stabilizer and, optionally, a multifunctional nucleophile monomer is prepared to form a water phase.

Stabilizer

According to the present invention, a stabilizer is added in the aqueous solution. In a particular embodiment, stabilizer is added in the aqueous solution to form the emulsion. According to an embodiment, the stabilizer is a colloidal stabilizer. According to an embodiment, the stabilizer is a polymeric stabilizer. The polymeric stabilizer is a polymer capable to stabilize oil/water interface as an emulsion. According to an embodiment, the stabilizer is a colloidal particle stabilizer. The colloidal particle stabilizer can form suspension in water phase and adsorb at the oil-water interface to stabilize the oil droplets (Pickering emulsion).

According to an embodiment, the stabilizer is an emulsifier. By emulsifier it meant a compound having both a polar group with an affinity for water (hydrophilic) and a nonpolar group with an affinity for oil (lipophilic). The hydrophilic part will dissolve in the water phase and the hydrophobic part will dissolve in the oil phase providing a film around droplets.

In a particular embodiment, the polymeric stabilizer is selected from the group consisting of polyvinyl alcohol, modified starch, polyvinyl pyrrolidone, anionic polysaccharides, acrylamide copolymer, inorganic particles, protein such as soy protein, rice protein, whey protein, white egg albumin, sodium caseinate, gelatin, bovine serum albumin, hydrolyzed soy protein, hydrolyzed sericin, pseudocollagen, silk protein, sericin powder, gum acacia, casein, sodium caseinate, soy (protein), hydrolyzed soy protein, pea protein, milk protein, whey protein, pectin, sugar beet pectin, sericin, bovine serum albumin, gelatin, and mixtures thereof

According to the present invention, the oil phase is added to the water phase to form an oil-in-water emulsion.

In a particular embodiment, the dispersion comprises between about 0.01% and 10.0% of at least stabilizer, percentage being expressed on a w/w basis relative to the total weight of the oil-in-water emulsion as obtained after step c) or d). In still another aspect of the invention, the dispersion comprises between about 0.02% and 5.0%, preferably between 0.05 and 3% of at least a stabilizer. In still another aspect of the invention, the dispersion comprises between about 0.1% and 3%, preferably between 0.1% and 2.0% by weight of at least a stabilizer.

The polymeric stabilizer is preferably comprised in an amount of 0.01 to 5.0 wt. %, preferably 0.05 to 3.0 wt. %, based on the total weight of the emulsion obtained after c) or d).

According to the present invention, conditions are applied to form a crosslinked polymeric shell via the polymerization of the multifunctional ethylenically unsaturated monomer, preferably the (meth)acrylate monomer and/or vinyl monomer, and the reaction between the multifunctional ethylenically unsaturated monomer, preferably the (meth)acrylate monomer and/or vinyl monomer, and the multifunctional nucleophile monomer.

Thereby, it is understood that reaction conditions have to be applied which allow a polymerization reaction between the ethylenically unsaturated functionalities of the multifunctional ethylenically unsaturated monomers, such as by radical polymerization, and a reaction between ethylenically unsaturated functionalities of the multifunctional ethylenically unsaturated monomers and the at least one nucleophilic functionality. In a particular embodiment, the reaction conditions are applied in a manner which allow a polymerization reaction between the ethylenically unsaturated functionalities of the multifunctional ethylenically unsaturated monomers, such as by radical polymerization, and at the same time a reaction between ethylenically unsaturated functionalities of the multifunctional ethylenically unsaturated monomers and the at least one nucleophilic functionality. In another particular embodiment, the reaction conditions are applied in a manner which allow a polymerization reaction between the ethylenically unsaturated functionalities of the multifunctional ethylenically unsaturated monomers, such as by radical polymerization, and at a different time, for example a later time, a polymerization reaction between ethylenically unsaturated functionalities of the multifunctional ethylenically unsaturated monomers and the at least one nucleophilic functionality.

In a particular embodiment, the conditions to form a crosslinked polymeric shell via the polymerization of the multifunctional ethylenically unsaturated monomer, preferably the multifunctional (meth)acrylate monomer and/or multifunctional vinyl monomer, in the presence of free-radicals and the reaction between a multifunctional ethylenically unsaturated monomer, preferably the multifunctional (meth)acrylate monomer and/or vinyl monomer, and a multifunction nucleophile monomer comprises the addition of a free-radical initiator, and/or a catalyst, preferably a base catalyst.

In case the reaction conditions are applied in a manner which allow a polymerization reaction between the ethylenically unsaturated functionalities of the multifunctional ethylenically unsaturated monomers, such as by radical polymerization, and at the same time a polymerization reaction between ethylenically unsaturated functionalities of the multifunctional ethylenically unsaturated monomers and the at least one nucleophilic functionality, the free-radical initiator and/or the catalyst, preferably the base catalyst may be added at the same time. In case the reaction conditions are applied in a manner which allow a polymerization reaction between the ethylenically unsaturated functionalities of the multifunctional ethylenically unsaturated monomers, such as by radical polymerization, and at a later time a polymerization reaction between ethylenically unsaturated functionalities of the multifunctional ethylenically unsaturated monomers and the at least one nucleophilic functionality the free-radical initiator and/or the catalyst, preferably the base catalyst may be added at the different times.

A free radical initiator is herein understood as a compound that can produce radical species and promote radical reactions. These substances generally possess weak bonds, i.e bonds that have small bond dissociation energies.

In a particular embodiment, the free radical initiator is a thermal radical initiator selecting from the group of organic peroxides, such as Benzoyl peroxide, Dicumyl peroxide, Di-tert-butyl peroxide, 2,5-Bis(tert-butylperoxy)-2,5-dimethylhexane, Cumene hydroperoxide, tert-Butyl hydroperoxide, 2,4-Pentanedione peroxide, 2-Butanone peroxide, Lauroyl peroxide, tert-Butyl peroxybenzoate, 2,5-Di(tert-butylperoxy)-2,5-dimethyl-3-hexyne, tert-Butyl peracetate, 1,1-Bis(tert-butylperoxy)cyclohexane; or selecting from the group of Azo compounds, such as 2,2′-Azobis(2-methylpropionitrile), 2,2′-Azobis(2-methylbutyronitrile), 2,2′-Azobis(2,4-dimethyl)valeronitrile, 4,4′-Azobis(4-cyanovaleric acid), Dimethyl 2,2′-azobis(2-methylpropionate), 1,1′-Azobis(cyclohexanecarbonitrile), 2,2′-Azobis(2-methylpropionamidine) dihydrochloride, 2,2′-Azobis[2-(2-imidazolin-2-yl)propane]Dihydrochloride; or selecting from the group of inorganic peroxides, such as Sodium persulfate, Potassium persulfate, Ammonium persulfate, Hydroxymethanesulfinic acid monosodium salt, Hydrogen peroxide. In a particular embodiment, the free radical initiator is a photo radical initiator selecting from the group of benzil and benzoin compounds, such as 4,4′-Dimethylbenzil, Benzoin, Benzoin methyl ether, 4,4′-Dimethoxybenzoin, Benzoin ethyl ether, Benzoin isopropyl ether, Benzoin isobutyl ether, Benzil, Benzil dimethylketal; or selecting from the group of acetophenone compounds, such as Acetophenone, 4-Ethoxyacetophenone, 2,2-Diethoxyacetophenone, 4-Acetophenol, 3-Acetophenol, 2,2-Dimethoxy-2-phenylacetophenone, 4′-tert-Butyl-2′,6′-dimethylacetophenone, 1-Hydroxycyclohexyl phenyl ketone, 2-Hydroxy-2-methylpropiophenone, 2-Hydroxy-4′-(2-hydroxyethoxy)-2-methylpropiophenone, 2-Benzyl-2-(dimethylamino)-4′-morpholinobutyrophenone, 4-Phenoxyacetophenone; or selecting from benzophenone compounds, such as Benzophenone, 4,4′-Bis(dimethylamino)benzophenone, 4,4′-Dihydroxybenzophenone, 2-Methylbenzophenone, 4,4′-Bis(diethylamino)benzophenone, 3-Methylbenzophenone, 4-Methylbenzophenone, 3,4-Dimethylbenzophenone, 4-(Dimethylamino)benzophenone, 4-Hydroxybenzophenone, 3-Hydroxybenzophenone, 4-Benzoylbiphenyl, Methyl benzoylformate, 4-Benzoylbenzoic acid, 2-Benzoylbenzoic acid, Methy-2-Benzoylbenzoate, 4-(Dimethylamino)benzophenone; or selecting from the group of thioxanthones compounds, such as Thioxanthen-9-one, 2,4-Diethylthioxanthone, Isopropylthioxanthone, N-Methylphenothiazine; or selecting from the group consisting of Camphorquinone, 2-tert-Butylanthraquinone, 9,10-Phenanthrenequinone, 1,4-Dibenzoylbenzene.

In a particular embodiment, the free radical initiator is a redox system which can produce radical species under mild condition. Examples of the redox system include hydrogen peroxide and a metal salt (such as ferrous ion, cuprous ion, cobalt ion), an organic peroxide (such as Benzoyl peroxide, Di-tert-butyl peroxide) and a metal salt (such as cuprous ion, cobalt ion), an organic peroxide and a tertiary amine, an inorganic peroxide (such as Sodium persulfate, Potassium persulfate, Ammonium persulfate) and ferrous ion, an inorganic peroxide and a sulfite, an inorganic peroxide and a thiosulfate.

The radical initiator is preferably comprised in an amount of 0.001 to 2.0 wt. %, preferably 0.001 to 1.0 wt. %, more preferably 0.001 to 0.5 wt. %, based on the total weight of the emulsion obtained after step c) or d).

A catalyst is herein understood a compound which is able of increasing the rate of a chemical reaction by its presence. A base catalyst is herein understood as a compound which is able of increasing the rate of a chemical reaction by its presence and which is a Broensted or Lewis base.

In a particular embodiment, the catalyst is a base catalyst, such as alkali metal hydroxides, alkali metal alkoxides, metal carbonate, alkali and alkali-earth metal oxide-based catalysts.

In a particular embodiment, the catalyst is a nucleophilic catalyst selecting from the group of Lewis bases, such as primary amines, secondary amines, tertiary amines, pyridine-based catalysts (e.g., Pyridine, 4-dimethylaminopyridine (DMAP), pyridonaphthyridine, 4-pyrrolidinopyridine (4-PPY)), amidine-based catalysts (e.g., 1,8-Diazabicyclo(5.4.0)undec-7-ene (DBU), 1,5-Diazabicyclo(4.3.0)non-5-ene (DBN)), imidazole, phosphine-based catalysts (e.g., Tri-n-butylphosphine, Tri-tert-butylphosphine).

In a particular embodiment, no catalyst is used during the reaction for crosslinked polymeric shell formation.

The catalyst is preferably comprised in an amount of 0.01 to 10.0 wt. %, preferably 0.02 to 5.0 wt. %, more preferably 0.05 to 3.0 wt. %, based on the total weight of the emulsion obtained after step c) or d).

In a particular embodiment, the functional equivalence ratio of (meth)acrylate groups or vinyl groups to thiol groups is higher than 1 to 1 (1:1), preferably higher than 2 to 1 (2:1), more preferably higher than 4 to 1 (4:1).

In a particular embodiment, the conditions applied to enhance cross-linking include a reaction at a temperature comprised between 5 and 90° C., preferably 10 to 80° C.

In a particular embodiment, the conditions applied to enhance cross-linking include a reaction in the presence of UV radiation.

In a particular embodiment, the conditions applied to enhance cross-linking include increased pressure, preferably a pressure higher than atmospheric pressure.

In a particular embodiment, the conditions applied to enhance cross-linking include a reaction time of 1 to 24 hours, preferably of 30 minutes to 8 hours.

In a particular embodiment, the conditions applied to enhance cross-linking include an inert gas protection during the reaction. In a more particular embodiment, the inert gas is nitrogen.

In a particular embodiment, at the end of step e) or during step e), one may also add to the invention's slurry a polymer selected from the group consisting of a non-ionic polysaccharide, a cationic polymer, a polysuccinimide derivative (as described for instance in WO2021185724) and mixtures thereof to form an outer coating to the microcapsule.

Non-ionic polysaccharide polymers are well known to a person skilled in the art and are described for instance in WO 2012/007438 page 29, lines 1 to 25 and in WO2013/026657 page 2, lines 12 to 19 and page 4, lines 3 to 12. Preferred non-ionic polysaccharides are selected from the group consisting of locust bean gum, xyloglucan, guar gum, hydroxypropyl guar, hydroxypropyl cellulose and hydroxypropyl methyl cellulose.

Cationic polymers are well known to a person skilled in the art. Preferred cationic polymers have cationic charge densities of at least 0.5 meq/g, more preferably at least about 1.5 meq/g, but also preferably less than about 7 meq/g, more preferably less than about 6.2 meq/g. The cationic charge density of the cationic polymers may be determined by the Kjeldahl method as described in the US Pharmacopoeia under chemical tests for Nitrogen determination. The preferred cationic polymers are chosen from those that contain units comprising primary, secondary, tertiary and/or quaternary amine groups that can either form part of the main polymer chain or can be borne by a side substituent directly connected thereto. The weight average (Mw) molecular weight of the cationic polymer is preferably between 10,000 and 3.5M Dalton, more preferably between 50,000 and 1.5M Dalton. According to a particular embodiment, one will use cationic polymers based on acrylamide, methacrylamide, N-vinylpyrrolidone, quatemized N,N-dimethylaminomethacrylate, diallyldimethylammonium chloride, quatemized vinylimidazole (3-methyl-1-vinyl-IH-imidazol-3-ium chloride), vinylpyrrolidone, acrylamidopropyltrimonium chloride, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. Preferably copolymers shall be selected from the group consisting of polyquatemium-5, polyquatemium-6, polyquaternium-7, polyquaterniumIO, polyquaternium-11, polyquatemium-16, polyquaternium-22, polyquatemium-28, polyquatemium-43, polyquaternium-44, polyquaternium-46, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. As specific examples of commercially available products, one may cite Salcare<®>SC60 (cationic copolymer of acrylamidopropyltrimonium chloride and acrylamide, origin: BASF) or Luviquat®, such as the PQ 11N, FC 550 or Style (polyquatemium-11 to 68 or quatemized copolymers of vinylpyrrolidone origin: BASF), or also the Jaguar® (C 13 S or Cl 7, origin Rhodia).

In a particular embodiment, there is added an amount of polymer described above comprised between about 0% and 5% w/w, or even between about 0.1% and 2% w/w, percentage being expressed on a w/w basis relative to the total weight of the emulsion as obtained after step c), or d) or the slurry of step e). It is clearly understood by a person skilled in the art that only part of said added polymers will be incorporated into/deposited on the microcapsule shell.

The multifunctional nucleophile monomer may be added in any one of steps (a), (b) or (d) of the present invention. The multifunctional nucleophilic monomer may be preferably added in one portion to one of the steps (a), (b) or (d).

The present invention also relates to a core-shell microcapsule slurry comprising at least one core-shell microcapsule, wherein the core-shell microcapsule comprises
an oil core comprising a hydrophobic material, preferably a perfume oil,
a cross-linked polymeric shell surrounding the oil core,
   wherein the cross-linked polymeric shell is obtained by a polymerization of a multifunctional ethylenically unsaturated monomer, preferably a multifunctional (meth)acrylate monomer and/or multifunctional vinyl monomer, and a reaction between multifunctional ethylenically unsaturated monomer, preferably a multifunctional (meth)acrylate monomer and/or multifunctional vinyl monomer, and a multifunctional nucleophile monomer.

According to the present invention, the core-shell microcapsule comprises an oil core comprising a hydrophobic material.

The oil core is based on the oil phase as described herein above and the definitions and embodiments herein-above apply mutatis mutandis to the oil core of the core-shell microcapsule comprised in the slurry.

In a particular embodiment, the oil core comprises a perfume.

According to the present invention, the core-shell microcapsule comprises a cross-linked polymeric shell surrounding the oil core, wherein the cross-linked polymeric shell is obtained by a polymerization of a multifunctional ethylenically unsaturated monomer, preferably a multifunctional (meth)acrylate monomer and/or multifunctional vinyl monomer, and a reaction between multifunctional ethylenically unsaturated monomer, preferably a multifunctional (meth)acrylate monomer and/or multifunctional vinyl monomer, and a multifunctional nucleophile monomer.

The cross-linked polymeric shell may be obtained by a polymerization from a multifunctional ethylenically unsaturated monomer and a reaction between multifunctional ethylenically unsaturated monomer and a multifunctional nucleophile monomer and corresponding definitions and embodiments as described herein-above apply mutatis mutandis to the cross-linked polymeric shell of the core-shell microcapsule comprised in the slurry.

In a particular embodiment, the core-shell microcapsules are isolated by drying the obtained core-shell microcapsule slurry. Drying can be achieved by submitting the obtained core-shell microcapsule slurry to a drying step, such as spray-drying, to provide the microcapsules as such, i.e. in a powdery form.

It is understood that any standard method known by a person skilled in the art to perform such drying is also applicable. In particular the slurry may be spray-dried preferably in the presence of a polymeric carrier material such as polyvinyl acetate, polyvinyl alcohol, dextrins, natural or modified starch, vegetable gums, pectins, xanthans, alginates, carragenans or cellulose derivatives to provide microcapsules in a powder form.

The present invention also relates to a core-shell microcapsule comprising
an oil core comprising a hydrophobic material, preferably a perfume oil,
a cross-linked polymeric shell surrounding the oil core and wherein the cross-linking polymer is obtained via the polymerization of a multifunctional ethylenically unsaturated monomer, preferably a multifunctional (meth)acrylate monomer and/or multifunctional vinyl monomer, and the reaction between a multifunctional ethylenically unsaturated monomer, preferably a multifunctional (meth)acrylate monomer and/or multifunctional vinyl monomer, and a multifunctional nucleophile monomer.

According to the present invention, the core-shell microcapsule comprises an oil core comprising a hydrophobic active material.

The oil core is based on the oil phase as described herein above and the definitions and embodiments herein-above apply mutatis mutandis to the oil core of the core-shell microcapsule.

In a particular embodiment, the oil core comprises a perfume.

According to the present invention, the core-shell microcapsule comprises a cross-linked polymeric shell surrounding the oil core, wherein the cross-linked polymeric shell is obtained by a polymerization of a multifunctional ethylenically unsaturated monomer, preferably a multifunctional (meth)acrylate monomer and/or multifunctional vinyl monomer, and a reaction between multifunctional ethylenically unsaturated monomer, preferably a multifunctional (meth)acrylate monomer and/or multifunctional vinyl monomer, and a multifunctional nucleophile monomer.

The cross-linked polymeric shell may be obtained by a polymerization from a multifunctional ethylenically unsaturated monomer and a reaction between multifunctional ethylenically unsaturated monomer and a multifunctional nucleophile monomer and corresponding definitions and embodiments herein-above apply mutatis mutandis to the cross-linked polymeric shell of the core-shell microcapsule.

In a particular embodiment, the shell material is a biodegradable material.

In a particular embodiment, the shell has a biodegradability of at least 40%, preferably at least 60%, 65%, 70%, 75%, 80%, 85%, 90%, 95% or 98%, within 60 days according to OECD301F.

In a particular embodiment, the core-shell microcapsule has a biodegradability of at least 40%, preferably at least 60%, preferably at least 65%, 70%, 75%, 80%, 85%, 90%, 95% or 98% within 60 days according to OECD301F.

Thereby it is understood that the core-shell microcapsule including all components, such as the core, shell and coating may have a biodegradability of at least 40%, preferably at least 60%, preferably at least 65%, 70%, 75%, 80%, 85%, 90%, 95% or 98% within 60 days according to OECD301F.

In a particular embodiment, the oil core, preferably perfume oil, has a biodegradability of at least 40%, preferably at least 60%, preferably at least 65%, 70%, 75%, 80%, 85%, 90%, 95% or 98% within 60 days according to OECD301F.

OECD301F is a standard test method on the biodegradability from the Organization of Economic Co-operation and Development.

A typical method for extracting the shell for measuring the biodegradability is disclosed in Gasparini and all in Molecules 2020, 25, 718.

According to an embodiment, the microcapsules of the invention (first type of microcapsule) can be used in combination with a second type of microcapsules.

Another object of the invention is a microcapsule delivery system comprising:
the microcapsules of the present invention as a first type of microcapsules, and
a second type of microcapsules, wherein the first type of microcapsules and the second type of microcapsules differ in their hydrophobic material and/or carrier material (shell or matrix) and/or in their coating material.

The microcapsules of the invention can be used in combination with active ingredients. An object of the invention is therefore a composition comprising:
(i) a core-shell microcapsule slurry as described herein-above or a core-shell microcapsule as described herein-above;
(ii) an active ingredient, preferably chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.

The present invention also relates to a perfuming composition comprising
a core-shell microcapsule slurry obtained by the method as described herein-above or as defined herein-above or a core-shell microcapsule obtained by the method as described herein-above or as defined herein-above,
at least one ingredient selected from the group consisting of a perfumery carrier and a perfumery base,
optionally, at least one perfumery adjuvant.

The perfuming composition may comprise the core-shell microcapsule slurry or core-shell microcapsule between 0.1 and 30 wt. %, based on the total weight of the perfuming composition.

The perfuming composition may further comprise an active ingredient. The active ingredient may preferably be chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.

In a particular embodiment, the perfuming composition comprises a free perfume oil.

By “free perfume” it is herein understood a perfume or perfume oil which is comprised in the perfuming composition and not entrapped in the core-shell microcapsule.

The perfuming composition may comprise the active ingredient, preferably the free perfume, between 0.1 and 30 wt. %, based on the total weight of the perfuming composition.

In a particular embodiment, the total amount of the microcapsule slurry or microcapsule is 0.05 to 5 wt. %, based on the total weight of the perfuming composition, and the total amount of the free perfume oil is 0.05 to 5 wt. %, based on the total weight of the perfuming composition.

In a particular embodiment, the total perfume oil of the perfume formulation entrapped in the core-shell microcapsule and total free perfume oil are present in the perfuming composition in a weight ratio of 1:20 to 20:1, preferably 10:1 to 1:10.

The perfuming composition can further comprise at least one perfuming co-ingredient and, optionally a perfumery adjuvant.

By “perfuming co-ingredient” it is herein understood a compound, which is used in a perfuming preparation or a composition to impart a hedonic effect and which is not a microcapsule as 20 defined above. In other words such a co-ingredient, to be considered as being a perfuming one, must be recognized by a person skilled in the art as being able to impart or modify in a positive or pleasant way the odor of a composition, and not just as having an odor. The nature and type of the perfuming co-ingredients present in the perfuming composition do not warrant a more detailed description here, which in any case would not be exhaustive, the skilled person being 25 able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-30 ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds (known as properfumes). Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

By “perfumery adjuvant” it is herein understood an ingredient capable of imparting additional 5 added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

According to an embodiment, the core-shell microcapsule slurry or core-shell microcapsule of the invention (first type of delivery system) can be used in combination with a second type of delivery system, preferably microcapsules.

Thus, according to a particular embodiment, the perfuming composition comprises:
the core-shell microcapsule slurry or core-shell microcapsule of the invention as a first type of delivery system, and
a second type of delivery system, wherein the first type of delivery system and the second type of delivery system differ in their perfuming formulations and/or carrier material (shell or matrix) and/or outer coating.

The core-shell microcapsule slurry or core-shell microcapsule of the present invention can advantageously be used in many application fields and used in perfumed consumer products.

The present invention also relates to a perfumed consumer product comprising
a core-shell microcapsule slurry obtained by the method a as described herein-above or as defined herein-above or a core-shell microcapsule obtained by the method as described herein-above or as defined herein-above and
a personal care, home care, or fabric care active base.

In a particular embodiment, the perfumed consumer product is selected from the group consisting of personal care composition, home care composition or fabric care composition, preferably in form of antiperspirants, hair care products, such as shampoo or hair-conditioner, body care products such as a shower gel, oral care products, laundry care products, preferably a detergent or a fabric softener.

Core-shell microcapsule slurries or core-shell microcapsules can be used in liquid form applicable to liquid consumer products as well as in powder form, applicable to powder consumer products.

The consumer products of the invention, can in particular be of used in perfumed consumer products such as product belonging to fine fragrance or “functional” perfumery. Functional perfumery is chosen in the group consisting of personal care composition, home care composition or fabric care composition, preferably in form of antiperspirants, hair care products, such as shampoo or hair-conditioner, body care products such as a shower gel, oral care products, laundry care products, preferably a detergent or a fabric softener.

In particular a liquid consumer product comprising:
from 2 to 65% by weight, relative to the total weight of the consumer product, of at least one surfactant;
water or a water-miscible hydrophilic organic solvent; and
a perfuming composition or core-shell microcapsule slurry or core-shell microcapsule as described herein above.

Also a powder consumer product comprising
from 2 to 65% by weight, relative to the total weight of the consumer product, of at least one surfactant; and
a perfuming composition or core-shell microcapsule slurry or core-shell microcapsule as described herein above.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumed consumer products can be a fine perfume, a splash or eau de perfume, a cologne, a shave or after-shave lotion, a liquid or solid detergent, a mono or multi chamber unit dose detergent, a fabric softener, a fabric refresher, liquid or solid scent-boosters (PEG/urea or salts), a dryer sheet, an ironing water, a paper, a bleach, a carpet cleaners, curtain-care products, a shampoo, a coloring preparation, a color care product, a hair shaping product, a dental care product, a disinfectant, an intimate care product, a hair spray, a hair conditioning product, a vanishing cream, a deodorant or antiperspirant, hair remover, tanning or sun product, nail products, skin cleansing, a makeup, a perfumed soap, shower or bath mousse, oil or gel, or a foot/hand care products, a hygiene product, an air freshener, a “ready to use” powdered air freshener, a mold remover, furnisher care, wipe, a dish detergent or hard-surface detergent, a leather care product, a car care product.

In a particular embodiment, the perfumed consumer product is a liquid or solid detergent, a fabric softener, liquid or solid scent-boosters (e.g. using PEG/urea or salts), a shampoo, a shower gel, a hair conditioning product (e.g. leave-on or rinse-off), a deodorant or antiperspirant.

Another object of the invention is a consumer product comprising:
a personal care active base, and
the core-shell microcapsule slurry or core-shell microcapsule as described herein above or the perfuming composition as defined above,
wherein the consumer product is in the form of a personal care composition.

Personal care active base in which the delivery system of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The personal care composition is preferably chosen in the group consisting of a hair-care product (e.g. a shampoo, hair conditioner, a colouring preparation or a hair spray), a cosmetic preparation (e.g. a vanishing cream, body lotion or a deodorant or antiperspirant), a skin-care product (e.g. a perfumed soap, shower or bath mousse, body wash, oil or gel, bath salts, or a hygiene product), oral care product (toothpaste or mouthwash composition) or a fine fragrance product (e.g. Eau de Toilette—EdT).

Another object of the invention is a consumer product comprising:
a home care or a fabric care active base, and
the core-shell microcapsule slurry or core-shell microcapsule as described herein above as defined above or the perfuming composition as defined above, wherein the consumer product is in the form of a home care or a fabric care composition.

Home care or fabric care bases in which the delivery system of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The home or fabric care composition is preferably chosen in the group consisting fabric softener, liquid detergent, powder detergent, liquid scent booster and solid scent booster.

For liquid consumer product mentioned below, by “active base”, it should be understood that the active base includes active materials (typically including surfactants) and water.

For solid consumer product mention below, by “active base”, it should be understood that the active base includes active materials (typically including surfactants) and auxiliary agents (such as bleaching agents, buffering agent; builders; soil release or soil suspension polymers; granulated enzyme particles, corrosion inhibitors, antifoaming, sud suppressing agents; dyes, fillers, and mixtures thereof).

### Fabric Softener

An object of the invention is a consumer product in the form of a fabric softener composition comprising:
a fabric softener active base; preferably comprising at least one active material chosen in the group consisting of dialkyl quaternary ammonium salts, dialkyl ester quaternary ammonium salts (esterquats), Hamburg esterquat (HEQ), TEAQ (triethanolamine quat), silicones and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Liquid Detergent

An object of the invention is a consumer product in the form of a liquid detergent composition comprising:
a liquid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Solid Detergent

An object of the invention is a consumer product in the form of a solid detergent composition comprising:
a solid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule powder or microcapsule slurry or microcapsules as defined above, preferably in an amount comprised between 0.05 to 15 wt %, more preferably between 0.1 and 5 wt % by weight based on the total weight of the composition,
optionally free perfume oil.

### Shampoo/Shower Gel

An object of the invention is a consumer product in the form of a shampoo or a shower gel composition comprising:
a shampoo or a shower gel active base; preferably comprising at least one active material chosen in the group consisting of sodium alkylether sulfate, ammonium alkylether sulfates, alkylamphoacetate, cocamidopropyl betaine, cocamide MEA, alkylglucosides and amino acid based surfactants and mixtures thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Rinse-Off Conditioner

An object of the invention is a consumer product in the form of a rinse-off conditioner composition comprising:
a rinse-off conditioner active base; preferably comprising at least one active material chosen in the group consisting of cetyltrimonium chloride, stearyl trimonium chloride, benzalkonium chloride, behentrimonium chloride and mixture thereof, the active base being used preferably in an amount comprised between 85 and 99.95% by weight based on the total weight of the composition,
a microcapsule slurry or microcapsules as defined above, preferably in an amount comprised between 0.05 to 15 wt %, more preferably between 0.1 and 5 wt % by weight based on the total weight of the composition,
optionally free perfume oil.

### Solid Scent Booster

An object of the invention is a consumer product in the form of a solid scent booster composition comprising:
a solid carrier, preferably chosen in the group consisting of urea, sodium chloride, sodium sulphate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, saccharides such as sucrose, mono-, di-, and polysaccharides and derivatives such as starch, cellulose, methyl cellulose, ethyl cellulose, propyl cellulose, polyols/sugar alcohols such as sorbitol, maltitol, xylitol, erythritol, and isomalt, PEG, PVP, citric acid or any water soluble solid acid, fatty alcohols or fatty acids and mixtures thereof,
a microcapsule slurry or microcapsules as defined above, in a powdered form, preferably in an amount comprised between 0.05 to 15 wt %, more preferably between 0.1 and 5 wt % by weight based on the total weight of the composition,
optionally free perfume oil.

### Liquid Scent Booster

An object of the invention is a consumer product in the form of a liquid scent booster composition comprising:
an aqueous phase,
a surfactant system essentially consisting of one or more than one non-ionic surfactant, wherein the surfactant system has a mean HLB between 10 and 14, preferably chosen in the group consisting of ethoxylated aliphatic alcohols, POE/PPG (polyoxyethylene and polyoxypropylene) ethers, mono and polyglyceryl esters, sucrose ester compounds, polyoxyethylene hydroxylesters, alkyl polyglucosides, amine oxides and combinations thereof;
a linker chosen in the group consisting of alcohols, salts and esters of carboxylic acids, salts and esters of hydroxyl carboxylic acids, fatty acids, fatty acid salts, glycerol fatty acids, surfactant having an HLB less than 10 and mixtures thereof, and
a microcapsule slurry or microcapsules as defined above, in the form of a slurry, preferably in an amount comprised between 0.05 to 15 wt %, more preferably between 0.1 and 5 wt % by weight based on the total weight of the composition,
optionally free perfume oil.

### Hair Coloration

An object of the invention is a consumer product in the form of an oxidative hair coloring composition comprising:
an oxidizing phase comprising an oxidizing agent and an alkaline phase comprising an alkakine agent, a dye precursor and a coupling compound; wherein said dye precursor and said coupling compound form an oxidative hair dye in the presence of the oxidizing agent, preferably in an amount comprised between 85 and 99.95% by weight based on the total weight of the composition,
microcapsules or microcapsules slurry as defined above, preferably in an amount comprised between 0.05 to 15 wt %, more preferably between 0.1 and 5 wt % by weight based on the total weight of the composition,
optionally free perfume oil

### Perfuming Composition

According to a particular embodiment, the consumer product is in the form of a perfuming composition comprising:
0.1 to 30%, preferably 0.1 to 20% of microcapsules or microcapsules slurry as defined previously,
0 to 40%, preferably 3-40% of perfume, and
20-90%, preferably 40-90% of ethanol, by weight based on the total weight of the perfuming composition.

The invention will now be further described by way of examples. It will be appreciated that the invention as claimed is not intended to be limited in any way by these examples.

### Examples of the Invention

### Example 1

i) Methacrylate monomer (EGDMA) and thiol (TMTP) were dissolved into oil as oil phase. Polymeric stabilizer was dissolved into DI water as water phase.
ii) O/W emulsion was formed by mixing oil phase with water phase with homogenizer, and the emulsion was degassed with N₂.
iii) Redox initiator (APS & NaHSO₃) was added into the emulsion, DBU was used as catalyst, and the reaction was carried out at RT for 2h and 80° C. for 1h under N₂ atmosphere.

TABLE 1
Ingredients of Example 1

Effective Amount (% wt)

| Ingredients | BM 1 | Microcapsule 1 |
|---|---|---|
| Gum Arabic ¹⁾ | 1.05 | 1.05 |
| Perfume oil | 27 | 26 |
| Ethylene glycol dimeth- | 3.0 | 3.0 |
| acrylate (EGDMA) | | |
| Trimethylolpropane tris(3- | / | 1.0 |
| mercaptopropionate) (TMTP) | | |
| Ammonium persulfate (APS) | 0.16 | 0.16 |
| Sodium bisulfite (NaHSO₃) | 0.05 | 0.05 |
| 1,8-Diazabicyclo[5.4.0]undec- | / | 0.12 |
| 7-ene (DBU) | | |

¹⁾ SuperstabTM (Origin: Nexira)

### Example 2

i) Acrylate monomer (PETA) and oil soluble initiator/catalyst (BPO) were dissolved into a perfume oil as oil phase. Polymeric stabilizer was dissolved into DI water as water phase.
ii) O/W emulsion was formed by mixing oil phase with water phase with homogenizer, and the emulsion was degassed with N₂.
iii) Thiol monomer (TMTP) and Redox initiator (APS & NaHSO₃) were added into the emulsion, and the reaction was carried out at RT for 2h and 80° C. for 1h under N₂ atmosphere.

TABLE 2
Ingredients of Example 2

Effective Amount (% wt)

| Ingredients | BM 2 | Microcapsule 2 |
|---|---|---|
| PVOH 18-88 ¹⁾ | 0.11 | 0.11 |
| Perfume oil | 26.84 | 26.84 |
| Pentaerythritol triacrylate | 3.0 | 3.0 |
| (PETA) | | |
| Trimethylolpropane tris(3- | / | 1.5 |
| mercaptopropionate) (TMTP) | | |
| Ammonium persulfate (APS) | 0.16 | 0.16 |
| Sodium bisulfite (NaHSO₃) | 0.05 | 0.05 |
| Benzoyl peroxide (BPO) | 0.16 | 0.16 |

¹⁾ Mowiol ® 18-88 (Origin: Sigma-Aldrich)

### Example 3

i) Methacrylate monomer (EGDMA), thiol functionalized Silane and oil soluble initiator/catalyst was dissolved into oil as oil phase. Polymeric stabilizer was dissolved into DI water as water phase.
ii) O/W emulsion was formed by mixing oil phase with water phase with homogenizer, and the emulsion was degassed with N₂.
iii) Redox initiator (APS & NaHSO₃) was added into the emulsion, and the reaction was carried out at RT for 2h and 80° C. for 1h under N₂ atmosphere.

TABLE 3
Ingredients of Example 3

| | Effective Amount (% wt) |
|---|---|
| Ingredients | Microcapsule 3 |
| PVOH 18-88 ¹⁾ | 0.11 |
| Perfume oil | 26.24 |
| Ethylene glycol dimeth- | 3.0 |
| acrylate (EGDMA) | |
| 3-Mercaptopropyltri- | 0.6 |
| methoxysilane | |
| Ammonium persulfate (APS) | 0.16 |
| Sodium bisulfite (NaHSO₃) | 0.05 |
| Benzoyl peroxide (BPO) | 0.16 |

¹⁾ Mowiol ® 18-88 (Origin: Sigma-Aldrich)

### Example 4

i) Acrylate monomer (TMPTA) and oil soluble initiator/catalyst (BPO) was dissolved into oil as oil phase. Polymeric stabilizer was dissolved into DI water as water phase.
ii) O/W emulsion was formed by mixing oil phase with water phase with homogenizer, and the emulsion was degassed with N₂.
iii) Redox initiator (APS & NaHSO₃) and amine (DETA) were added into the emulsion, and the reaction was carried out at RT for 2h and 80° C. for 2h under N₂ atmosphere.

TABLE 4
Ingredients of Example 4

| | Effective Amount (% wt) |
|---|---|
| Ingredients | Microcapsule 4 |
| PVOH 18-88 ¹⁾ | 0.11 |
| Perfume oil | 25.09 |
| Trimethylolpropane triacrylate | 2.83 |
| (TMPTA) | |
| Diethylenetriamine (DETA) | 0.49 |
| Ammonium persulfate (APS) | 0.15 |
| Sodium bisulfite (NaHSO₃) | 0.05 |
| Benzoyl peroxide (BPO) | 0.16 |

¹⁾ Mowiol ® 18-88 (Origin: Sigma-Aldrich)

### Example 5

i) Methacrylate monomer (EGDMA), amino-Silane and oil soluble initiator/catalyst (BPO) was dissolved into oil as oil phase. Polymeric stabilizer was dissolved into DI water as water phase.
ii) O/W emulsion was formed by mixing oil phase with water phase with homogenizer, and the emulsion was degassed with N₂.
iii) Redox initiator (APS & NaHSO₃) was added into the emulsion, and the reaction was carried out at RT for 2h and 50° C. for 4h under N₂ atmosphere.

TABLE 5
Ingredients of Example 5

| | Effective Amount (% wt) |
|---|---|
| Ingredients | Microcapsule 5 |
| PVOH 18-88 ¹⁾ | 0.11 |
| Perfume oil | 24.32 |
| Ethylene glycol | 2.90 |
| dimethacrylate (EGDMA) | |
| 3-Aminopropyl(diethoxy) | 1.40 |
| methylsilane | |
| Ammonium persulfate (APS) | 0.16 |
| Sodium bisulfite (NaHSO₃) | 0.05 |
| Benzoyl peroxide (BPO) | 0.17 |

¹⁾ Mowiol ® 18-88 (Origin: Sigma-Aldrich)

### Example 6

iv) Acrylate monomer (PETA), amino-Silane and oil soluble initiator/catalyst (BPO) was dissolved into oil as oil phase. Polymeric stabilizer was dissolved into DI water as water phase.
v) O/W emulsion was formed by mixing oil phase with water phase with homogenizer, and the emulsion was degassed with N₂.
vi) Redox initiator (APS & NaHSO₃) was added into the emulsion, and the reaction was carried out at RT for 2h and 50° C. for 4h under N₂ atmosphere.

TABLE 6
Ingredients of Example 6

| | Effective Amount (% wt) |
|---|---|
| Ingredients | Microcapsule 6 |
| PVOH 18-88 ¹⁾ | 0.11 |
| Perfume oil | 24.30 |
| Pentaerythritol triacrylate | 2.91 |
| (PETA) | |
| 3-Aminopropyl(diethoxy) | 1.39 |
| methylsilane | |
| Ammonium persulfate (APS) | 0.15 |
| Sodium bisulfite (NaHSO₃) | 0.05 |
| Benzoyl peroxide (BPO) | 0.16 |

### Example 7

i) Methacrylate monomer (EGDMA) was dissolved into oil as oil phase. Polymeric stabilizer was dissolved into DI water as water phase.
ii) O/W emulsion was formed by mixing oil phase with water phase with homogenizer, and the emulsion was degassed with N₂.
iii) Redox initiator (APS & NaHSO₃), Acetoacetate (CAA) and catalyst (DBU) were added into the emulsion, and the reaction was carried out at RT for 2h and 80° C. for 3h under N₂ atmosphere.

TABLE 7
Ingredients of Example 7

| | Effective Amount (% wt) |
|---|---|
| Ingredients | Microcapsule 7 |
| PVOH 18-88 ¹⁾ | 0.11 |
| Perfume oil | 25.14 |
| Ethylene glycol | 2.84 |
| dimethacrylate (EGDMA) | |
| Cellulose Acetoacetate | 0.76 |
| (CAA) | |
| Ammonium persulfate (APS) | 0.15 |
| Sodium bisulfite (NaHSO₃) | 0.05 |
| 1,8-Diazabicyclo | 0.15 |
| [5.4.0]undec-7-ene (DBU) | |

### Example 8

i) Methacrylate monomer (EGDMA) was dissolved into oil as oil phase. Polymeric stabilizer was dissolved into DI water as water phase.
ii) O/W emulsion was formed by mixing oil phase with water phase with homogenizer, and the emulsion was degassed with N₂.
iii) Redox initiator (APS & NaHSO₃), Acetoacetate (SAA) and catalyst (DBU) were added into the emulsion, and the reaction was carried out at RT for 2h and 80° C. for 3h under N₂ atmosphere.

TABLE 8
Ingredients of Example 8

| Ingredients | Effective Amount (% wt) |
|---|---|
| Gum Arabic ¹⁾ | 0.81 |
| Perfume oil | 20.77 |
| Ethylene glycol | 2.31 |
| dimethacrylate (EGDMA) | |
| Starch Acetoacetate (SAA) | 0.16 |
| Ammonium persulfate (APS) | 0.12 |
| Sodium bisulfite (NaHSO₃) | 0.04 |
| 1,8-Diazabicyclo | 0.15 |
| [5.4.0]undec-7-ene (DBU) | |

¹⁾ SuperstabTM (Origin: Nexira)

### Example 9: Perfumery Composition

Pure fragrance ingredient was encapsulated, such as (2-tert-butylcyclohexyl) acetate) and (4-tert-butyl-1-cyclohexyl acetate), which are not react with the acrylate and thiol.

### Example 10: Performance Determination

i) Size measurement: Mean size (D [4,3]) of microcapsule slurry was measured by using the Mastersizer 3000 equipment from Malvern Instruments Ltd., UK
ii) Zeta potential measurement: Zeta potential of microcapsules was examined using Zetasizer Nano ZS from Malvern Instruments Ltd., UK
iii) Storage stability: storage stability of microcapsules was measured by dispersing microcapsule slurry into a softener base (see table 10) with a perfume oil dosage in base at 0.2% and stored at 37° C. for 3 days. The leaked perfume from microcapsule into base was extracted by a solvent and quantified by a GC-MS.

### a) Results

TABLE 9
Results of the the performance determination

| Sample | Mean size | Zeta potential | Oil leakage |
|---|---|---|---|
| BM 1 | No stable microcapsule was obtained | | |
| | (capsule broken in heating) | | |
| Microcapsule 1 | 25.4 μm | −41.9 ± 0.6 mV | 10.9% |
| BM 2 | 21.8 μm | −10.2 ± 1.8 mV | >90% |
| Microcapsule 2 | 23.9 μm | −34.6 ± 0.9 mV | 15.0% |
| Microcapsule 3 | 36.8 μm | −5.3 ± 2.6 mV | 11.2% |

### b) Conclusion

The microcapsules with dual crosslinked shell are more stable and display low permeability in a softener base.

### Example 11: Fabric Conditioner Composition

Microcapsule slurry (see examples 1 to 8) are dispersed in a fabric conditioner (softener) base described in Table below to obtain a concentration of encapsulated perfume oil at 0.2%.

TABLE 10
Fabric conditioner composition

| Product | Origin | Wt % |
|---|---|---|
| Stepantex VL 90A | | 8.88 |
| Calcium Chloride Sol. 10% | | 0.36 |
| Proxel GXL | Avecia | 0.04 |
| Perfume | Firmenich SA | 1 |
| Water | | 89.72 |
| TOTAL | | 100 |

### Example 12: Liquid Detergent Composition

Microcapsule slurry (see examples 1 to 8) is dispersed in a liquid detergent base described in Table below to obtain a concentration of encapsulated perfume oil at 0.22%.

TABLE 11
Liquid detergent composition

| Ingredients | Concentration [wt %] |
|---|---|
| Sodium C14-17 Alkyl Sec Sulfonate¹⁾ | 7 |
| Fatty acids, C12-18 and C18-unsaturated²⁾ | 7.5 |
| C12/14 fatty alcohol polyglycol ether with 7 | 17 |
| mol EO³⁾ | |
| Triethanolamine | 7.5 |
| Propylene Glycol | 11 |
| Citric acid | 6.5 |
| Potassium Hydroxyde | 9.5 |
| Protease | 0.2 |
| Amylase | 0.2 |
| Mannanase | 0.2 |
| Acrylates/Steareth-20 Methacrylate | 6 |
| structuring Crosspolymer⁴⁾ | |
| Deionized Water | 27.4 |

¹⁾Hostapur SAS 60; Origin: Clariant
²⁾Edenor K 12-18; Origin: Cognis
³⁾Genapol LA 070; Origin: Clariant
⁴⁾Aculyn 88; Origin: Dow Chemical

### Example 13: Rinse-Off Conditioner

Microcapsule slurry (see examples 1 to 8) is dispersed in a rinse-off conditioner base described in table below to obtain a concentration of encapsulated perfume oil at 0.5%.

TABLE 12
Rinse-off conditioner composition

| | | Concentration |
|---|---|---|
| | Ingredients | [wt %] |
| A | Water deionized | 81.8 |
| | Behentrimonium Chloride ¹⁾ | 2.5 |
| | Hydroxyethylcellulose ²⁾ | 1.5 |
| B | Cetearyl Alcohol ³⁾ | 4 |
| | Glyceryl Stearate (and) PEG-100 Stearate ⁴⁾ | 2 |
| | Behentrimonium Methosulfate (and) Cetyl alcohol | 4 |
| | (and) Butylene Glycol ⁵⁾ | |
| | Ethoxy (20) Stearyl Alcohol ⁶⁾ | 1 |
| C | Amodimethicone (and) Trideceth-12 (and) | 3 |
| | Cetrimonium Chloride ⁷⁾ | |
| | Chlorhexidine Digluconate ⁸⁾ 20% aqueous solution | 0.2 |
| D | Citric acid 10% aqueous sol. till pH 3.5-4 | q.s. |
| | TOTAL: | 100 |

¹⁾ Genamin KDM P, Clariant
²⁾ Tylose H10 Y G4, Shin Etsu
³⁾ Lanette O, BASF
⁴⁾ Arlacel 165-FP-MBAL-PA-(RB), Croda
⁵⁾ Incroquat Behenyl TMS-50-MBAL-PA-(MH) HA4112, Croda
⁶⁾ SP Brij S20 MBAL-PA(RB), Croda
⁷⁾ Xiameter DC MEM-0949 Emulsion, Dow Corning
⁸⁾ Alfa Aesar

### Example 14: Shampoo Composition

Microcapsule slurry (see examples 1 to 8) is weighed and mixed in a shampoo composition to add the equivalent of 0.2% perfume.

TABLE 13
Shampoo composition

| | | Concentration |
|---|---|---|
| | Ingredients | [wt %] |
| A | Water deionized | 44.4 |
| | Polyquaternium-10 ¹⁾ | 0.3 |
| | Glycerin 85% ²⁾ | 1 |
| | DMDM Hydantoin ³⁾ | 0.2 |
| B | Sodium Laureth Sulfate ⁴⁾ | 28 |
| | Cocamidopropyl Betaine ⁵⁾ | 3.2 |
| | Disodium Cocoamphodiacetate ⁶⁾ | 4 |
| | Ethoxy (20) Stearyl Alcohol ⁶⁾ | 1 |
| C | Sodium Laureth Sulfate ⁴⁾ | 3 |
| | Glyceryl Laureate ⁷⁾ | 0.2 |
| D | Water deionized | 1 |
| | Sodium Methylparaben ⁸⁾ | 0.1 |
| E | Sodium Chloride 10% aqueous sol. | 15 |
| | Citric acid 10% aqueous sol. till pH 5.5-6 | q.s. |
| | Perfume | 0.5 |
| | TOTAL: | 100 |

¹⁾ Ucare Polymer JR-400, Noveon
²⁾ Schweizerhall
³⁾ Glydant, Lonza
⁴⁾ Texapon NSO IS, Cognis
⁵⁾ Tego Betain F 50, Evonik
⁶⁾ Amphotensid GB 2009, Zschimmer & Schwarz
⁷⁾ Monomuls 90 L-12, Gruenau
⁸⁾ Nipagin Monosodium, NIPA

### Example 15: Antiperspirant Roll-on Emulsion Composition

Microcapsule slurry (see examples 1 to 8) is weighed and mixed in antiperspirant roll-on emulsion composition to add the equivalent of 0.2% perfume.

TABLE 14
Antiperspirant composition

| | Amount |
|---|---|
| Ingredient | (wt %) |
| Steareth-2¹⁾ (Part A) | 3.25 |
| Steareth-21²⁾ (Part A) | 0.75 |
| PPG-15 Stearyl Ether³⁾ (Part A) | 4 |
| WATER deionised (Part B) | 51 |
| Aluminum Chlorohydrate 50% | 40 |
| aqueous solution⁴⁾ (Part C) | |
| Fragrance (Part D) | 1 |

¹⁾BRIJ 72; origin: ICI
²⁾BRIJ 721; origin: ICI
³⁾ARLAMOL E; origin: UNIQEMA-CRODA
⁴⁾LOCRON L; origin: CLARIAN

Part A and B are heated separately to 75° C.; Part A is added to Part B under stirring and the mixture is homogenized for 10 min. Then, the mixture is cooled under stirring; and Part C is slowly added when the mixture reached 45° C. and Part D when the mixture reached at 35° C. while stirring. Then the mixture is cooled to room temperature.

### Example 16: Shower-Gel Composition

Microcapsule slurry (see examples 1 to 8) is weighed and mixed in the following composition to add the equivalent of 0.2% perfume.

TABLE 15
Shower gel composition

| | Amount | |
|---|---|---|
| Ingredients | (% wt) | Function |
| WATER deionised | 49.350 | Solvent |
| Tetrasodium EDTA ¹⁾ | 0.050 | Chelating agent |
| Acrylates Copolymer²⁾ | 6.000 | Thickener |
| Sodium C12-C15 Pareth Sulfate ³⁾ | 35.000 | Surfactant |
| Sodium Hydroxide 20% aqueous solution | 1.000 | pH adjuster |
| Cocamidopropyl Betaine⁴⁾ | 8.000 | Surfactant |
| Methylchloroisothiazolinone and | 0.100 | Preservative |
| Methylisothiazolinone⁵⁾ | | |
| Citric Acid (40%) | 0.500 | pH adjuster |

¹⁾ EDETA B POWDER; trademark and origin: BASF
²⁾CARBOPOL AQUA SF-1 POLYMER; trademark and origin: NOVEON
³⁾ ZETESOL AO 328 U; trademark and origin: ZSCHIMMER & SCHWARZ
⁴⁾TEGO-BETAIN F 50; trademark and origin: GOLDSCHMIDT
⁵⁾KATHON CG; trademark and origin: ROHM & HASS.

1. A method of preparing a core-shell microcapsule slurry, wherein the process comprises the steps of:
a. Dissolving a multifunctional ethylenically unsaturated monomer, and, optionally, a multifunctional nucleophile monomer in an oil phase comprising a hydrophobic material, to form an oil phase,
b. Preparing an aqueous solution of a stabilizer and, optionally, a multifunctional nucleophile monomer to form a water phase,
c. Adding the oil phase to the water phase to form an oil-in-water emulsion,
d. Optionally, adding a multifunctional nucleophile monomer to the oil-in-water emulsion, and
e. Applying conditions to form a cross-linked polymeric shell via the polymerization of the multifunctional ethylenically unsaturated monomer, and the reaction between the multifunctional ethylenically unsaturated monomer, wherein the multifunctional nucleophile monomer is added at least in one of steps (a), (b) or (d).

2. The method according to claim 1, wherein the multifunctional ethylenically unsaturated monomer is a multifunctional (meth)acrylate monomer.

3. The method according to claim 1, wherein the multifunctional ethylenically unsaturated monomer is a multifunctional vinyl monomer.

4. The method according to claim 1, wherein the multifunctional (meth)acrylate monomer is selected from the group consisting of pentaerythritol tri(meth)acrylate, trimethylolpropane tri(meth)acrylate, pentaerytrithol tetra(meth)acrylate, Tetra(ethylene glycol) di(meth)acrylate, dipentaerytrhitol penta(meth)acryalate, dipentaerytrithol hexa(meth)acrylate, tricyclodecane dimenthanol di(meth)acrylate, ethylene glycol di(meth)acrylate, di(ethylene glycol) di(meth)acrylate, triethylene glycol di(meth)acrylate, propylene glycol di(meth)acrylate, 1,4-butanediol di(meth)acrylate, 1,6-hexanediol di(meth)acrylate, glycerol di(meth)acrylate, 1,3-butanediol di(meth)acrylate, neopentyl glycol di(meth)acrylate, 1,10-decanediol di(meth)acrylate, pentaerythritol tri(meth)acrylate, pentaerythritol tetra(meth)acrylate, dipentaerythritol hexa(meth)acrylate, triallylformal tri(meth)acrylate, allyl methacrylate, trimethylol propane tri(meth)acrylate, tributanediol di(eth)acrylate, PEG 200 di(meth)acrylate, PEG 400 di(meth)acrylate, PEG 600 di(eth)acrylate, pentaerythritol-tetraacrylate, pentaerythritol triacrylate (PETIA), 1,4-butanediol diacrylate (BDA-2), ethylene glycol di methacrylate, trimethylolpropane triacrylate, hexane diol diacrylate, ((2,4,6-trioxocyclohexane-1,3,5-triyl)tris(oxy))tris(ethane-2,1-diyl) triacrylate, tris(2-acryloyloxyethyl) Isocyanurate, 1,3,5-triacryloylhexahydro-1,3,5-triazine, bis[2-(meth)acryloyloxyethyl] phosphate, bis[glyceryl di(meth)acrylate] phosphate, urethane acrylate oligomers with two to six acrylate groups, polyester/polyether acrylates with more than two acrylate groups, epoxy acrylates with more than two acrylate groups, and mixtures thereof.

5. The method according to claim 1, wherein the multifunctional vinyl monomer is selected from the group consisting of diethylene glycol divinyl ether, 1,5-hexadiene, divinyl adipate, diallyl phthalate, 2,4,6-trimethyl-2,4,6-trivinylcyclotrisiloxane, triallyl phosphate, diallylamine, allyl sulfide, 1,3-divinyltetramethyldisiloxane, divinyl sulfone, tetraallyloxyethane, 1,3,5-trivinyl-1,1,3,5,5-pentamethyltrisiloxane, diallyl isophthalate, allyl ether, triallyl isocyanurate, 1,3-diisopropenylbenzene, 2,2-bis(allyloxymethyl)-1-butanol, diethyl diallylmalonate, 1,2,4-trivinylcyclohexane, triallylamine, diallyl adipate, triallyl cyanurate, diallyl maleate, diallyl terephthalate, 1,3-diisopropenylbenzene, diallyl 1,4-cyclohexanedicarboxylate, bis(vinylsulfonyl)methane, 1,4-cyclohexanedimethanol divinyl ether, di(ethylene glycol) divinyl ether, tri(ethylene glycol) divinyl ether, and mixtures thereof.

6. The method according to claim 1, wherein the multifunctional nucleophile monomer is multifunctional thiol, multifunctional amine or multifunctional acetoacetate, comprising two or more groups selected from the group consisting of thiols, amines, acetoacetates, and mixtures thereof.

7. The method according to claim 1, wherein the multifunctional nucleophile monomer is a silane comprising at least one nucleophilic group.

8. The method according to claim 1, wherein the multifunctional nucleophile monomer is a multifunctional thiol selected from the group consisting of multifunctional thiol monomers, trimethylolpropane tris(3)-mercaptopropionate), 1,6-hexanedithiol, 2,2′-thiodiethanethiol, 2-amino-1,3,5-triazine-4,6-dithiol, 1,3-propanedithiol, 1,4-butanedithiol, 2,2′-(ethylenedioxy)diethanethiol, benzene-1,4-dithiol, toluene-3,4-dithiol, ethylene glycol bis-mercaptoacetate, ethylene bis(3-mercaptopropionate), 1,4-butanediol bis(thioglycolate), dithiothreitol, pentaerythritol tetra (3-mercaptopropionate), tris[2-(3-mercaptopropionyloxy)ethyl] isocyanurate, trimethylolpropane tris(thioglycolate), thiosilane monomers, 3-mercaptopropyltrimethoxysilane, 3-mercaptopropyltriethoxysilane, (3-mercaptopropyl)methyldimethoxysilane, 11-mercaptoundecyltrimethoxysilane, and mixtures thereof.

9. The method according to claim 1, wherein the stabilizer is a polymer stabilizer.

10. The method according to claim 1, wherein the conditions to form a cross-linked polymeric shell via the polymerization of the multifunctional ethylenically unsaturated monomer in the presence of free-radicals and the reaction between a multifunctional ethylenically unsaturated monomer and a multifunction nucleophile monomer comprises the addition of a free-radical initiator, and/or a catalyst.

11. The method according to claim 1, wherein the functional equivalence ratio of (meth)acrylate groups or vinyl groups to thiol groups is higher than 1 to 1 (1:1).

12. A core-shell microcapsule slurry comprising at least one core-shell microcapsule, wherein the core-shell microcapsule comprises
an oil core comprising a hydrophobic material, and
a cross-linked polymeric shell surrounding the oil core,
wherein the cross-linked polymeric shell is obtained by a polymerization of a multifunctional ethylenically unsaturated monomer and a reaction between a multifunctional ethylenically unsaturated monomer.

13. A core-shell microcapsule comprising
an oil core comprising a hydrophobic material, and
a cross-linked polymeric shell surrounding the oil core and wherein the cross-linking polymer is obtained via the polymerization of a multifunctional ethylenically unsaturated monomer, and a reaction between a multifunctional ethylenically unsaturated monomer and a multifunctional nucleophile monomer.

14. A perfuming composition comprising
a core-shell microcapsule slurry obtained by the method according to claim 1,
at least one ingredient selected from the group consisting of a perfumery carrier and a perfumery base, and
optionally, at least one perfumery adjuvant.

15. A perfumed consumer product comprising
a core-shell microcapsule slurry obtained by the method according to claim 1, and
a personal care, home care, or fabric care active base.

16. The method according to claim 1, wherein the hydrophobic material is a perfume oil.

17. The method according to claim 1, wherein the multifunctional nucleophile monomer is a silane comprising at least one nucleophilic group selected from the group consisting of thiol, amine, and acetoacetate.

18. The method according to claim 1, wherein the polymer stabilizer is selected from the group consisting of polyvinyl alcohol, polyvinyl pyrrolidone, gum acacia, casein, sodium caseinate, soy (protein), hydrolyzed soy protein, pea protein, milk protein, whey protein, pectin, sugar beet pectin, sericin, bovine serum albumin, gelatin, and mixtures thereof.

19. The method according to claim 1, wherein the functional equivalence ratio of (meth)acrylate groups or vinyl groups to thiol groups is higher than 2 to 1 (2:1).

20. The method according to claim 1, wherein the functional equivalence ratio of (meth)acrylate groups or vinyl groups to thiol groups is higher than 4 to 1 (4:1).
